SEQUENCE LISTING (2 sequences)

SEQ ID NO: 1 - DNA, 996 bp, Human
atgacgcccaacagcactggcgaggtgcccagccccattcccaagggggctttggggctctccctggccctggcaagcctcatcatcaccgcgaacctgctcctagccctgggcatcgcctgggaccgccgcctgcgcagcccacctgctggctgcttcttcctgagcctactgctggctgggctgctcacgggtctggcattgcccacattgccagggctgtggaaccagagtcgccggggttactggtcctgcctcctcgtctacttggctcccaacttctccttcctctccctgcttgccaacctcttgctggtgcacggggagcgctacatggcagtcctgaggccactccagccccctgggagcattcggctggccctgctcctcacctgggctggtcccctgctctttgccagtctgcccgctctggggtggaaccactggacccctggtgccaactgcagctcccaggctatcttcccagccccctacctgtacctcgaagtctatgggctcctgctgcccgccgtgggtgctgctgccttcctctctgtccgcgtgctggccactgcccaccgccagctgcaggacatctgccggctggagcgggcagtgtgccgcgatgagccctccgccctggcccgggcccttacctggaggcaggcaagggcacaggctggagccatgctgctcttcgggctgtgctgggggccctacgtggccacactgctcctctcagtcctggcctatgagcagcgcccgccactggggcctgggacactgttgtccctcctctccctaggaagtgccagtgcagcggcagtgcccgtagccatggggctgggcgatcagcgctacacagccccctggagggcagccgcccaaaggtgcctgcaggggctgtggggaagagcctcccgggacagtcccggccccagcattgcctaccacccaagcagccaaagcagtgtcgacctggacttgaactaaagg

SEQ ID NO: 2 - protein, 330 aa, Human
MTPNSTGEVPSPIPKGALGLSLALASLIITANLLLALGIAWDRRLRSPPAGCFFLSLLLAGLLTGLALPTLPGLWNQSRRGYWSCLLVYLAPNFSFLSLLANLLLVHGERYMAVLRPLQPPGSIRLALLLTWAGPLLFASLPALFWNHWTPGANCSSQAIFPAPYLYLEVYGLLLPAVGAAAFLSVRVLATAHRQLQDICRLERAVCRDEPSALARALTWRQARAQAGAMLLFGLCWGPYVATLLLSVLAYEQRPPLGPGTLLSLLSLGSASAAAVPVAMGLGDQRYTAPWRAAAQRCLQGLWGRASRDSPGPSIAYHPSSQSSVDLDLN

(19) United States
(12) Patent Application Publication
(10) Pub. No.: US 2004/0214237 A1
(43) Pub. Date: Oct. 28, 2004

(54) DNA encoding SNORF138 and uses thereof

(75) Inventors: Roman Artymyshyn, Flemington, NJ (US); Liliana Delgado, Rochelle Park, NJ (US); Pierre J. Vaysse, Franklin Lakes, NJ (US); Hong Zhang, Fort Lee, NJ (US); Weinong Guo, Marietta, GA (US)

Correspondence Address: John P. White, Cooper & Dunham LLP, 1185 Avenue of the Americas, New York, NY, 10036, US

(73) Assignee: Synaptic Pharmaceutical Corporation

(21) Appl. No.: 10/679,185

(22) Filed: Oct. 3, 2003

Related U.S. Application Data
(60) Provisional application No. 60/415,743, filed on Oct. 3, 2002.

(51) Int. Cl. G01N 33/53; G01N 33/567

(52) U.S. Cl. 435/7.2

(57) ABSTRACT

This invention provides methods of treating an abnormality that is linked to the activity of the mammalian SNORF138 receptor, as well as methods of determining binding of compounds to the mammalian SNORF138 receptor, methods of identifying agonists and antagonists of the SNORF138 receptor, and agonists and antagonists so identified.

This application claims the benefit of U.S. Provisional Application No. 60/415,743, filed on Oct. 3, 2002, the contents of which is hereby incorporated by reference.

### BACKGROUND OF THE INVENTION

Throughout this application various publications are referred to by partial citations within parenthesis. Full citations for these publications may be found at the end of the specification immediately preceding the claims. The disclosures of these publications, in their entireties, are hereby incorporated by reference into this application in order to more fully describe the state of the art to which the invention pertains.

G-protein coupled receptors (GPCRs) represent a major class of cell surface receptors with which many ligands interact to mediate their effects. GPCRs are characterized by seven membrane-spanning domains interconnected with three intracellular loops and three extracellular loops. Ligand binding to its cognate receptor results in the activation of the G proteins coupled to the receptor. This marks the beginning of a biochemical cascade that results in the production of second messengers such as cAMP or accumulation of calcium in the cytosol. While structural motifs that characterize a GPCR can be recognized in the predicted amino acid sequence of a novel receptor, an endogenous ligand that activates the GPCR cannot always be predicted from its primary structure. Thus, a novel receptor sequence may be designated as a GPCR when it possesses the characteristic structural motif, even though an endogenous ligand for the receptor has not yet been defined. GPCRs can also be activated by ligands other than an endogenous ligand.

Receptor ligands comprise a diverse group of molecules that subserve or modulate communication between cells. They include, but are not limited to, neuropeptides, amino acids, biogenic amines, lipids and lipid metabolites, other metabolic byproducts, and synthetic molecules. Many of these ligands interact with specific cell surface receptors such as GPCRs which transduce signals from the extracellular space to the intracellular space (cytosol) of the cell. The identification of activating ligands which mimic the effects of an endogenous ligand allows both the characterization of the receptor signal transduction pathway and the development of methods for screening for compounds (agonists or antagonists) that activate or block receptor function. Such agonists or antagonists permit determination the biochemical role of the receptor in normal and pathological states, and thus therapeutic potential of drugs that would act at the receptor. Finally, the use of such agonists accelerates the discovery of an endogenous ligand through iterative structure/function analyses and datamining. The present invention describes methods for identifying compounds that bind to, bind to and activate, or bind to and inhibit the activation of the GPCR designated SNORF138. The present invention identifies a SNORF138 agonist. Finally, the present invention describes the use of SNORF138 agonists and/or antagonists in the treatment of disorders, including, but not limited to, immune disorders and inflammatory disease.

### SUMMARY OF THE INVENTION

This invention provides a process for identifying a chemical compound which specifically binds to a mammalian SNORF138 receptor which comprises contacting cells containing DNA encoding, and expressing on their cell surface, the mammalian SNORF138 receptor, wherein such cells do not normally express the mammalian SNORF138 receptor, with the compound under conditions suitable for binding, and detecting specific binding of the chemical compound to the mammalian SNORF138 receptor.

Furthermore, this invention provides a process for identifying a chemical compound which specifically binds to a mammalian SNORF138 receptor which comprises contacting a membrane preparation from cells containing DNA encoding, and expressing on their cell surface, the mammalian SNORF138 receptor, wherein such cells do not normally express the mammalian SNORF138 receptor, with the compound under conditions suitable for binding, and detecting specific binding of the chemical compound to the mammalian SNORF138 receptor.

Moreover, this invention provides a process involving competitive binding for identifying a chemical compound which specifically binds to a mammalian SNORF138 receptor which comprises separately contacting cells expressing on their cell surface the mammalian SNORF138 receptor, wherein such cells do not normally express the mammalian SNORF138 receptor, with both the chemical compound and a second chemical compound known to bind to the receptor, and with only the second chemical compound, under conditions suitable for binding of such compounds to the receptor, and detecting specific binding of the chemical compound to the mammalian SNORF138 receptor, a decrease in the binding of the second chemical compound to the mammalian SNORF138 receptor in the presence of the chemical compound being tested indicating that such chemical compound binds to the mammalian SNORF138 receptor.

This invention also provides a process involving competitive binding for identifying a chemical compound which specifically binds to a mammalian SNORF138 receptor which comprises separately contacting a membrane preparation from cells expressing on their cell surface the mammalian SNORF138 receptor, wherein such cells do not normally express the mammalian SNORF138 receptor, with both the chemical compound and a second chemical compound known to bind to the receptor, and with only the second chemical compound, under conditions suitable for binding of such compounds to the receptor, and detecting specific binding of the chemical compound to the mammalian SNORF138 receptor, a decrease in the binding of the second chemical compound to the mammalian SNORF138 receptor in the presence of the chemical compound being tested indicating that such chemical compound binds to the mammalian SNORF138 receptor.

This invention further provides a method of screening a plurality of chemical compounds not known to bind to a mammalian SNORF138 receptor to identify a compound which specifically binds to the mammalian SNORF138 receptor, which comprises (a) contacting cells transfected with, and expressing, DNA encoding the mammalian SNORF138 receptor with a compound known to bind specifically to the mammalian SNORF138 receptor; (b) contacting the cells of step (a) with the plurality of compounds not known to bind specifically to the mammalian SNORF138 receptor, under conditions permitting binding of compounds known to bind to the mammalian SNORF138 receptor; (c) determining whether the binding of the compound known to bind to the mammalian SNORF138 receptor is reduced in the presence of the plurality of compounds, relative to the binding of the compound in the absence of the plurality of compounds; and if so (d) separately determining the binding to the mammalian SNORF138 receptor of each compound included in the plurality of compounds, so as to thereby identify any compound included therein which specifically binds to the mammalian SNORF138 receptor.

This invention still further provides a method of screening a plurality of chemical compounds not known to bind to a mammalian SNORF138 receptor to identify a compound which specifically binds to the mammalian SNORF138 receptor, which comprises (a) contacting a membrane preparation from cells transfected with, and expressing, DNA encoding the mammalian SNORF138 receptor with the plurality of compounds not known to bind specifically to the mammalian SNORF138 receptor under conditions permitting binding of compounds known to bind to the mammalian SNORF138 receptor; (b) determining whether the binding of a compound known to bind to the mammalian SNORF138 receptor is reduced in the presence of the plurality of compounds, relative to the binding of the compound in the absence of the plurality of compounds; and if so (c) separately determining the binding to the mammalian SNORF138 receptor of each compound included in the plurality of compounds, so as to thereby identify any compound included therein which specifically binds to the mammalian SNORF138 receptor.

This invention also provides a process for determining whether a chemical compound is a mammalian SNORF138 receptor agonist which comprises contacting cells transfected with and expressing DNA encoding the mammalian SNORF138 receptor with the compound under conditions permitting the activation of the mammalian SNORF138 receptor, and detecting any increase in mammalian SNORF138 receptor activity, so as to thereby determine whether the compound is a mammalian SNORF138 receptor agonist.

This invention further provides a process for determining whether a chemical compound is a mammalian SNORF138 receptor antagonist which comprises contacting cells transfected with and expressing DNA encoding the mammalian SNORF138 receptor with the compound in the presence of a known mammalian SNORF138 receptor agonist, under conditions permitting the activation of the mammalian SNORF138 receptor, and detecting any decrease in mammalian SNORF138 receptor activity, so as to thereby determine whether the compound is a mammalian SNORF138 receptor antagonist.

Moreover, this invention provides a process for determining whether a chemical compound specifically binds to and activates a mammalian SNORF138 receptor, which comprises contacting cells producing a second messenger response and expressing on their cell surface the mammalian SNORF138 receptor, wherein such cells do not normally express the mammalian SNORF138 receptor, with the chemical compound under conditions suitable for activation of the mammalian SNORF138 receptor, and measuring the second messenger response in the presence and in the absence of the chemical compound, a change in the second messenger response in the presence of the chemical compound indicating that the compound activates the mammalian SNORF138 receptor.

This invention further provides a process for determining whether a chemical compound specifically binds to and inhibits activation of a mammalian SNORF138 receptor, which comprises separately contacting cells producing a second messenger response and expressing on their cell surface the mammalian SNORF138 receptor, wherein such cells do not normally express the mammalian SNORF138 receptor, with both the chemical compound and a second chemical compound known to activate the mammalian SNORF138 receptor, and with only the second chemical compound, under conditions suitable for activation of the mammalian SNORF138 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the mammalian SNORF138 receptor.

This invention provides a method of screening a plurality of chemical compounds not known to activate a mammalian SNORF138 receptor to identify a compound which activates the mammalian SNORF138 receptor which comprises: (a) contacting cells transfected with and expressing the mammalian SNORF138 receptor with the plurality of compounds not known to activate the mammalian SNORF138 receptor, under conditions permitting activation of the mammalian SNORF138 receptor; (b) determining whether the activity of the mammalian SNORF138 receptor is increased in the presence of one or more of the compounds; and if so (c) separately determining whether the activation of the mammalian SNORF138 receptor is increased by any compound included in the plurality of compounds, so as to thereby identify each compound which activates the mammalian SNORF138 receptor.

This invention further provides a method of screening a plurality of chemical compounds not known to inhibit the activation of a mammalian SNORF138 receptor to identify a compound which inhibits the activation of the mammalian SNORF138 receptor, which comprises: (a) contacting cells transfected with and expressing the mammalian SNORF138 receptor with the plurality of compounds in the presence of a known mammalian SNORF138 receptor agonist, under conditions permitting activation of the mammalian SNORF138 receptor; (b) determining whether the extent or amount of activation of the mammalian SNORF138 receptor is reduced in the presence of one or more of the compounds, relative to the extent or amount of activation of the mammalian SNORF138 receptor in the absence of such one or more compounds; and if so (c) separately determining whether each such compound inhibits activation of the mammalian SNORF138 receptor for each compound included in the plurality of compounds, so as to thereby identify any compound included in such plurality of compounds which inhibits the activation of the mammalian SNORF138 receptor.

This invention additionally provides a method of treating an abnormality in a subject wherein the abnormality is alleviated by increasing the activity of a mammalian SNORF138 receptor which comprises administering to the subject a compound which is a mammalian SNORF138 receptor agonist in an amount effective to treat the abnormality.

This invention further provides a method of treating an abnormality in a subject wherein the abnormality is alleviated by decreasing the activity of a mammalian SNORF138 receptor which comprises administering to the subject a compound which is a mammalian SNORF138 receptor antagonist in an amount effective to treat the abnormality.

This invention provides an isolated nucleic acid encoding a mammalian SNORF138 receptor.

This invention further provides a purified mammalian SNORF138 receptor protein.

Furthermore, this invention provides a nucleic acid probe comprising at least 15 nucleotides, which probe specifically hybridizes with a nucleic acid encoding a mammalian SNORF138 receptor, wherein the probe has a sequence complementary to a unique sequence present within one of the two strands of the nucleic acid encoding the human SNORF138 receptor contained in plasmid MSP70-hSNORF138-f (ATCC Patent Deposit Designation PTA-4725).

This invention provides a nucleic acid probe comprising at least 15 nucleotides, which probe specifically hybridizes with a nucleic acid encoding a mammalian SNORF138 receptor, wherein the probe has a sequence complementary to a unique sequence present within (a) the nucleic acid sequence shown in FIGS. 1A-1B (SEQ ID NO: 1) or (b) the reverse complement thereof.

Furthermore, this invention provides a method for diagnosing a predisposition to a disorder associated with the activity of a specific mammalian allele which comprises: (a) obtaining DNA of subjects suffering from the disorder; (b) performing a restriction digest of the DNA with a panel of restriction enzymes; (c) electrophoretically separating the resulting DNA fragments on a sizing gel; (d) contacting the resulting gel with a nucleic acid probe capable of specifically hybridizing with a unique sequence included within the sequence of a nucleic acid molecule encoding a mammalian SNORF138 receptor and labeled with a detectable marker; (e) detecting labeled bands which have hybridized to the DNA encoding a mammalian SNORF138 receptor of claim 1 to create a unique band pattern specific to the DNA of subjects suffering from the disorder; (f) repeating steps (a)-(e) with DNA obtained for diagnosis from subjects not yet suffering from the disorder; and (g) comparing the unique band pattern specific to the DNA of subjects suffering from the disorder from step (e) with the band pattern from step (f) for subjects not yet suffering from the disorder so as to determine whether the patterns are the same or different and thereby diagnose predisposition to the disorder if the patterns are the same.

This invention further provides a transgenic, nonhuman mammal comprising a homologous recombination knockout of the native mammalian SNORF138 receptor.

### BRIEF DESCRIPTION OF THE FIGURES

FIGS. 1A-1B Nucleotide sequence including the sequence encoding a human SNORF138 receptor (SEQ ID NO: 1). The putative open reading frame is indicated by underlining the start (ATG) codon (at positions 1-3) and the stop codon (at positions 991-993).

In addition, a partial 3′ untranslated sequence is shown.

FIGS. 2A-2B Deduced amino acid sequence (SEQ ID NO: 2) of the human SNORF138 receptor encoded by the open reading frame indicated in the nucleotide sequence shown in FIGS. 1A-1B (SEQ ID NO: 1). The seven putative transmembrane (TM) regions are underlined.

FIG. 3

Betamethasone valerate-induced calcium-activated chloride currents in Xenopus laevis oocytes expressing human SNORF138 (hSNORF138).

A. Electrophysiological response to 50 M betamethasone valerate from a voltage clamped oocyte injected with only cDNA encoding CFTR. Arrows X and Y indicate the bullet application of vehicle and betamethasone valerate, respectively. (Representative of 4 separate experiments.)

B. Electrophysiological response to 50 M betamethasone valerate from a voltage clamped oocyte injected with cDNA encoding hSNOF138 and cDNA encoding CFTR. Arrows X and Y indicate the bullet application of vehicle and betamethasone valerate, respectively. (Representative of 8 separate experiments.)

### DETAILED DESCRIPTION OF THE INVENTION

This invention provides for a process for identifying a chemical compound which specifically binds to a mammalian SNORF138 receptor which comprises contacting cells transfected with DNA encoding, and expressing on their cell surface, the mammalian SNORF138 receptor, wherein such cells prior to being transfected with such DNA do not normally express the mammalian SNORF138 receptor, with the compound under conditions suitable for binding, and detecting specific binding of the chemical compound to the mammalian SNORF138 receptor.

This invention further provides for a process for identifying a chemical compound which specifically binds to a mammalian SNORF138 receptor which comprises contacting a membrane preparation from cells transfected with DNA encoding and expressing on their cell surface the mammalian SNORF138 receptor, wherein such cells prior to being transfected with such DNA do not normally express the mammalian SNORF138 receptor, with the compound under conditions suitable for binding, and detecting specific binding of the chemical compound to the mammalian SNORF138 receptor.

This invention still further provides a process involving competitive binding for identifying a chemical compound which specifically binds to a mammalian SNORF138 receptor which comprises separately contacting cells transfected with DNA encoding and expressing on their cell surface the mammalian SNORF138 receptor, wherein such cells prior to being transfected with such DNA do not normally express the mammalian SNORF138 receptor, with both the chemical compound and a second chemical compound known to bind to the receptor, and with only the second chemical compound, under conditions suitable for binding of such compounds to the receptor, and detecting specific binding of the chemical compound to the mammalian SNORF138 receptor, a decrease in the binding of the second chemical compound to the mammalian SNORF138 receptor in the presence of the chemical compound being tested indicating that such chemical compound binds to the mammalian SNORF138 receptor.

This invention provides a process involving competitive binding for identifying a chemical compound which specifically binds to a mammalian SNORF138 receptor which comprises separately contacting a membrane preparation from cells transfected with DNA encoding and expressing on their cell surface the mammalian SNORF138 receptor, wherein such cells prior to being transfected with such DNA do not normally express the mammalian SNORF138 receptor, with both the chemical compound and a second chemical compound known to bind to the receptor, and with only the second chemical compound, under conditions suitable for binding of such compounds to the receptor, and detecting specific binding of the chemical compound to the mammalian SNORF138 receptor, a decrease in the binding of the second chemical compound to the mammalian SNORF138 receptor in the presence of the chemical compound being tested indicating that such chemical compound binds to the mammalian SNORF138 receptor.

In an embodiment of the present invention, the second chemical compound is a glucocorticosteroid. In an embodiment of the present invention, the second chemical compound is betamethasone valerate.

In an embodiment, the mammalian SNORF138 receptor is a human SNORF138 receptor. In another embodiment, the mammalian SNORF138 receptor has substantially the same amino acid sequence as the human SNORF138 receptor encoded by plasmid MSP70-hSNORF138-f (ATCC Patent Deposit Designation PTA-4725).

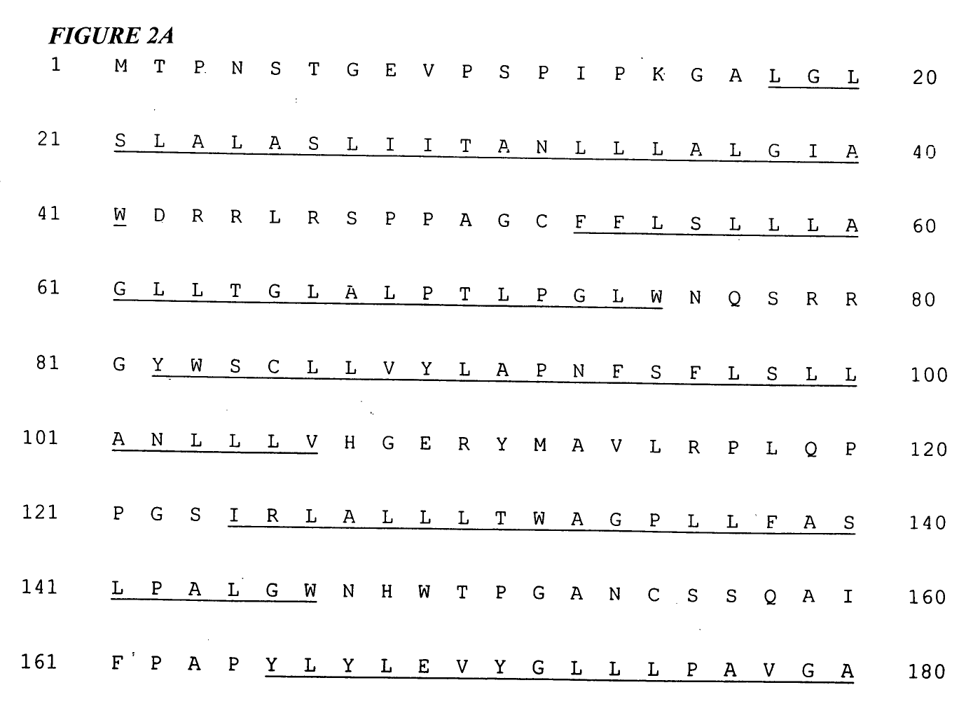

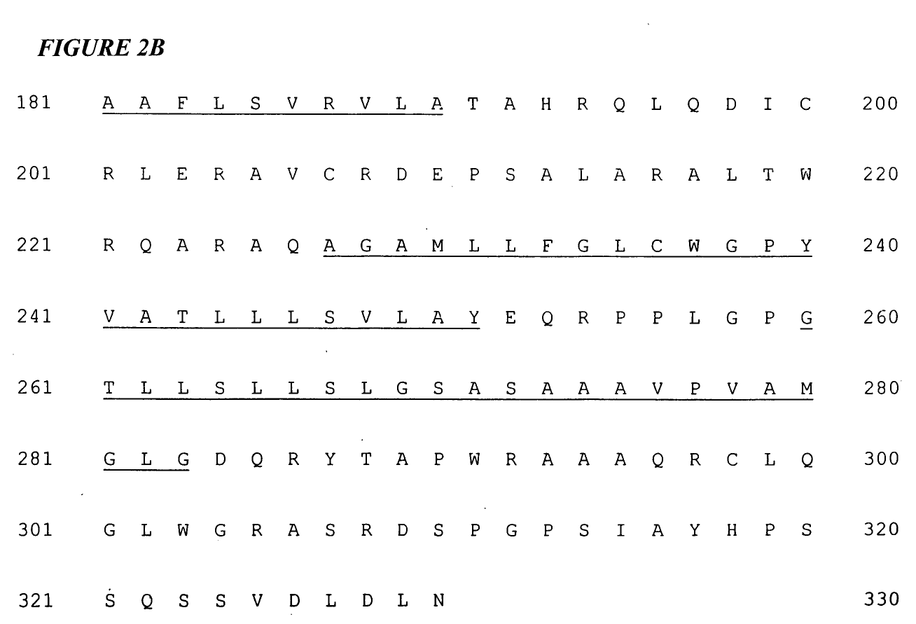

In another embodiment, the mammalian SNORF138 receptor has substantially the same amino acid sequence as that shown in FIGS. 2A-2B (SEQ ID NO: 2). In another embodiment, the mammalian SNORF138 receptor has the amino acid sequence shown in FIGS. 2A-2B (SEQ ID NO: 2).

In one embodiment, the compound is not previously known to bind to a mammalian SNORF138 receptor. In one embodiment, the cell is an insect cell. In one embodiment, the cell is a mammalian cell. In another embodiment, the cell is normeuronal in origin. In another embodiment, the normeuronal cell is a COS-7 cell, a human embryonic kidney cell, a CHO cell, a NIH-3T3 cell, a mouse Y1 cell, or a LM(tk-) cell. In another embodiment, the compound is a compound not previously known to bind to a mammalian SNORF138 receptor. This invention provides a compound identified by the preceding processes according to this invention.

Methods for preparing transfected cells and membrane preparations from such cells are described hereinafter.

This invention provides for a method of screening a plurality of chemical compounds not known to bind to a mammalian SNORF138 receptor to identify a compound which specifically binds to the mammalian SNORF138 receptor, which comprises (a) contacting cells transfected with, and expressing, DNA encoding the mammalian SNORF138 receptor with a compound known to bind specifically to the mammalian SNORF138 receptor; (b) contacting the cells of step (a) with the plurality of compounds not known to bind specifically to the mammalian SNORF138 receptor, under conditions permitting binding of compounds known to bind to the mammalian SNORF138 receptor; (c) determining whether the binding of the compound known to bind to the mammalian SNORF138 receptor is reduced in the presence of the plurality of compounds, relative to the binding of the compound in the absence of the plurality of compounds; and if so (d) separately determining the binding to the mammalian SNORF138 receptor of each compound included in the plurality of compounds, so as to thereby identify any compound included therein which specifically binds to the mammalian SNORF138 receptor.

This invention provides a method of screening a plurality of chemical compounds not known to bind to a mammalian SNORF138 receptor to identify a compound which specifically binds to the mammalian SNORF138 receptor, which comprises (a) contacting a membrane preparation from cells transfected with, and expressing, DNA encoding the mammalian SNORF138 receptor with the plurality of compounds not known to bind specifically to the mammalian SNORF138 receptor under conditions permitting binding of compounds known to bind to the mammalian SNORF138 receptor; (b) determining whether the binding of a compound known to bind to the mammalian SNORF138 receptor is reduced in the presence of the plurality of compounds, relative to the binding of the compound in the absence of the plurality of compounds; and if so (c) separately determining the binding to the mammalian SNORF138 receptor of each compound included in the plurality of compounds, so as to thereby identify any compound included therein which specifically binds to the mammalian SNORF138 receptor.

Furthermore, this invention provides a process for determining whether a chemical compound is a mammalian SNORF138 receptor agonist which comprises contacting cells transfected with and expressing DNA encoding the mammalian SNORF138 receptor with the compound under conditions permitting the activation of the mammalian SNORF138 receptor, and detecting any increase in mammalian SNORF138 receptor activity, so as to thereby determine whether the compound is a mammalian SNORF138 receptor agonist.

This invention further provides a process for determining whether a chemical compound is a mammalian SNORF138 receptor agonist which comprises contacting cells transfected with and expressing DNA encoding the mammalian SNORF138 receptor with the compound under conditions permitting the activation of the mammalian SNORF138 receptor, and detecting any increase in mammalian SNORF138 receptor activity, so as to thereby determine whether the compound is a mammalian SNORF138 receptor agonist.

This invention also provides a process for determining whether a chemical compound is a mammalian SNORF138 receptor antagonist which comprises contacting cells transfected with and expressing DNA encoding the mammalian SNORF138 receptor with the compound in the presence of a known mammalian SNORF138 receptor agonist, under conditions permitting the activation of the mammalian SNORF138 receptor, and detecting any decrease in mammalian SNORF138 receptor activity, so as to thereby determine whether the compound is a mammalian SNORF138 receptor antagonist.

This invention also provides a process for determining whether a chemical compound is a mammalian SNORF138 receptor antagonist which comprises contacting cells transfected with and expressing DNA encoding the mammalian SNORF138 receptor with the compound in the presence of a known mammalian SNORF138 receptor agonist, under conditions permitting the activation of the mammalian SNORF138 receptor, and detecting any decrease in mammalian SNORF138 receptor activity, so as to thereby determine whether the compound is a mammalian SNORF138 receptor antagonist.

In an embodiment, the mammalian SNORF138 receptor is a human SNORF138 receptor. In another embodiment, the mammalian SNORF138 receptor has substantially the same amino acid sequence as the human SNORF138 receptor encoded by plasmid MSP70-hSNORF138-f (ATCC Patent Deposit Designation PTA-4725).

In another embodiment, the mammalian SNORF138 receptor has substantially the same amino acid sequence as that shown in FIGS. 2A-2B (SEQ ID NO: 2). In another embodiment, the mammalian SNORF138 receptor has the amino acid sequence shown in FIGS. 2A-2B (SEQ ID NO: 2).

In one embodiment, the compound is not previously known to bind to a mammalian SNORF138 receptor. In one embodiment, the cell is an insect cell. In one embodiment, the cell is a mammalian cell. In another embodiment, the cell is normeuronal in origin. In another embodiment, the normeuronal cell is a COS-7 cell, a human embryonic kidney cell, a CHO cell, a NIH-3T3 cell, a mouse Y1 cell, or a LM(tk-) cell. In another embodiment, the compound is a compound not previously known to bind to a mammalian SNORF138 receptor. This invention provides a compound identified by the preceding processes according to this invention.

Methods for preparing transfected cells and membrane preparations from such cells are described hereinafter.

This invention still further provides a composition, for example a pharmaceutical composition, which comprises an amount of a mammalian SNORF138 receptor agonist determined by a process according to this invention effective to increase activity of a mammalian SNORF138 receptor and a carrier, for example, a pharmaceutically acceptable carrier. In one embodiment, the mammalian SNORF138 receptor agonist is not previously known.

Also, this invention provides a composition, for example a pharmaceutical composition, which comprises an amount of a mammalian SNORF138 receptor antagonist determined by a process according to this invention effective to reduce activity of a mammalian SNORF138 receptor and a carrier, for example, a pharmaceutically acceptable carrier. Also, this invention provides a composition, for example a pharmaceutical composition, which comprises an amount of a mammalian SNORF138 receptor antagonist determined by a process according to this invention effective to reduce activity of a mammalian SNORF138 receptor and a carrier, for example, a pharmaceutically acceptable carrier.

In one embodiment, the mammalian SNORF138 receptor antagonist is not previously known. In an embodiment, the mammalian SNORF138 receptor antagonist is a human SNORF138 receptor antagonist.

This invention moreover provides a process for determining whether a chemical compound specifically binds to and activates a mammalian SNORF138 receptor, which comprises contacting cells producing a second messenger response and expressing on their cell surface the mammalian SNORF138 receptor, wherein such cells do not normally express the mammalian SNORF138 receptor, with the chemical compound under conditions suitable for activation of the mammalian SNORF138 receptor, and measuring the second messenger response in the presence and in the absence of the chemical compound, a change, e.g. an increase, in the second messenger response in the presence of the chemical compound indicating that the compound activates the mammalian SNORF138 receptor.

In one embodiment, the second messenger response comprises chloride channel activation and the change in second messenger is an increase in the level of chloride current.

In another embodiment, the second messenger response comprises change in intracellular calcium levels and the change in second messenger is an increase in the measure of intracellular calcium. In another embodiment, the second messenger response comprises release of inositol phosphate and the change in second messenger is an increase in the level of inositol phosphate. In another embodiment, the second messenger response comprises release of arachidonic acid and the change in second messenger is an increase in the level of arachidonic acid. In yet another embodiment, the second messenger response comprises GTP S ligand binding and the change in second messenger is an increase in GTP S ligand binding. In another embodiment, the second messenger response comprises activation of MAP kinase and the change in second messenger response is an increase in MAP kinase activation. In a further embodiment, the second messenger response comprises cAMP accumulation and the change in second messenger response is a reduction in cAMP accumulation.

This invention still further provides a process for determining whether a chemical compound specifically binds to and inhibits activation of a mammalian SNORF138 receptor, which comprises separately contacting cells producing a second messenger response and expressing on their cell surface the mammalian SNORF138 receptor, wherein such cells do not normally express the mammalian SNORF138 receptor, with both the chemical compound and a second chemical compound known to activate the mammalian SNORF138 receptor, and with only the second chemical compound, under conditions suitable for activation of the mammalian SNORF138 receptor, and measuring the second messenger response in the presence of only the second chemical compound and in the presence of both the second chemical compound and the chemical compound, a smaller change, e.g. increase, in the second messenger response in the presence of both the chemical compound and the second chemical compound than in the presence of only the second chemical compound indicating that the chemical compound inhibits activation of the mammalian SNORF138 receptor.

In an embodiment of the present invention, the second chemical compound is a glucocorticosteroid. In an embodiment of the present invention, the second chemical compound is betamethasone valerate.

In one embodiment, the second messenger response comprises chloride channel activation and the change in second messenger response is a smaller increase in the level of chloride current in the presence of both the chemical compound and the second chemical compound than in the presence of only the second chemical compound. In another embodiment, the second messenger response comprises change in intracellular calcium levels and the change in second messenger response is a smaller increase in the measure of intracellular calcium in the presence of both the chemical compound and the second chemical compound than in the presence of only the second chemical compound. In another embodiment, the second messenger response comprises release of inositol phosphate and the change in second messenger response is a smaller increase in the level of inositol phosphate in the presence of both the chemical compound and the second chemical compound than in the presence of only the second chemical compound.

In one embodiment, the second messenger response comprises activation of MAP kinase and the change in second messenger response is a smaller increase in the level of MAP kinase activation in the presence of both the chemical compound and the second chemical compound than in the presence of only the second chemical compound. In another embodiment, the second messenger response comprises change in cAMP levels and the change in second messenger response is a smaller change in the level of cAMP in the presence of both the chemical compound and the second chemical compound than in the presence of only the second chemical compound. In another embodiment, the second messenger response comprises release of arachidonic acid and the change in second messenger response is an increase in the level of arachidonic acid levels in the presence of both the chemical compound and the second chemical compound than in the presence of only the second chemical compound. In a further embodiment, the second messenger response comprises GTP S ligand binding and the change in second messenger is a smaller increase in GTP S ligand binding in the presence of both the chemical compound and the second chemical compound than in the presence of only the second chemical compound.

In an embodiment, the mammalian SNORF138 receptor is a human SNORF138 receptor. In another embodiment, the mammalian SNORF138 receptor has substantially the same amino acid sequence as the human SNORF138 receptor encoded by plasmid MSP70-hSNORF138-f (ATCC Patent Deposit Designation PTA-4725).

In another embodiment, the mammalian SNORF138 receptor has substantially the same amino acid sequence as that shown in FIGS. 2A-2B (SEQ ID NO: 2). In another embodiment, the mammalian SNORF138 receptor has the amino acid sequence shown in FIGS. 2A-2B (SEQ ID NO: 2).

In one embodiment, the compound is not previously known to bind to a mammalian SNORF138 receptor. In one embodiment, the cell is an insect cell. In one embodiment, the cell is a mammalian cell. In another embodiment, the cell is normeuronal in origin. In another embodiment, the normeuronal cell is a COS-7 cell, a human embryonic kidney cell, a CHO cell, a NIH-3T3 cell, a mouse Y1 cell, or a LM(tk-) cell. In another embodiment, the compound is a compound not previously known to bind to a mammalian SNORF138 receptor. This invention provides a compound identified by the preceding processes according to this invention.

Methods for preparing transfected cells and membrane preparations from such cells are described hereinafter.

Further, this invention provides a compound determined by a process according to this invention and a composition, for example, a pharmaceutical composition, which comprises an amount of a mammalian SNORF138 receptor agonist determined to be such by a process according to this invention effective to increase activity of a mammalian SNORF138 receptor and a carrier, for example, a pharmaceutically acceptable carrier. In one embodiment, the mammalian SNORF138 receptor agonist is not previously known.

This invention also provides a composition, for example, a pharmaceutical composition, which comprises an amount of a mammalian SNORF138 antagonist determined to be such by a process according to this invention, effective to reduce activity of the mammalian SNORF138 receptor and a carrier, for example a pharmaceutically acceptable carrier. In one embodiment, the mammalian SNORF138 antagonist is not previously known.

This invention yet further provides a method of screening a plurality of chemical compounds not known to activate a mammalian SNORF138 receptor to identify a compound which activates the mammalian SNORF138 receptor which comprises: (a) contacting cells transfected with and expressing the mammalian SNORF138 receptor with the plurality of compounds not known to activate the mammalian SNORF138 receptor, under conditions permitting activation of the mammalian SNORF138 receptor; (b) determining whether the activity of the mammalian SNORF138 receptor is increased in the presence of one or more of the compounds; and if so (c) separately determining whether the activation of the mammalian SNORF138 receptor is increased by any compound included in the plurality of compounds, so as to thereby identify each compound which activates the mammalian SNORF138 receptor.

This invention provides a method of screening a plurality of chemical compounds not known to inhibit the activation of a mammalian SNORF138 receptor to identify a compound which inhibits the activation of the mammalian SNORF138 receptor, which comprises: (a) contacting cells transfected with and expressing the mammalian SNORF138 receptor with the plurality of compounds in the presence of a known mammalian SNORF138 receptor agonist, under conditions permitting activation of the mammalian SNORF138 receptor; (b) determining whether the extent or amount of activation of the mammalian SNORF138 receptor is reduced in the presence of one or more of the compounds, relative to the extent or amount of activation of the mammalian SNORF138 receptor in the absence of such one or more compounds; and if so (c) separately determining whether each such compound inhibits activation of the mammalian SNORF138 receptor for each compound included in the plurality of compounds, so as to thereby identify any compound included in such plurality of compounds which inhibits the activation of the mammalian SNORF138 receptor.

In an embodiment, the mammalian SNORF138 receptor is a human SNORF138 receptor. In another embodiment, the mammalian SNORF138 receptor has substantially the same amino acid sequence as the human SNORF138 receptor encoded by plasmid MSP70-hSNORF138-f (ATCC Patent Deposit Designation PTA-4725).

In another embodiment, the mammalian SNORF138 receptor has substantially the same amino acid sequence as that shown in FIGS. 2A-2B (SEQ ID NO: 2). In another embodiment, the mammalian SNORF138 receptor has the amino acid sequence shown in FIGS. 2A-2B (SEQ ID NO: 2).

In one embodiment, the compound is not previously known to bind to a mammalian SNORF138 receptor. In one embodiment, the cell is an insect cell. In one embodiment, the cell is a mammalian cell. In another embodiment, the cell is normeuronal in origin. In another embodiment, the normeuronal cell is a COS-7 cell, a human embryonic kidney cell, a CHO cell, a NIH-3T3 cell, a mouse Y1 cell, or a LM(tk-) cell. In another embodiment, the compound is a compound not previously known to bind to a mammalian SNORF138 receptor. This invention provides a compound identified by the preceding processes according to this invention.

Methods for preparing transfected cells and membrane preparations from such cells are described hereinafter.

This invention also provides a composition, for example, a pharmaceutical composition, comprising a compound identified by a method according to this invention in an amount effective to increase mammalian SNORF138 receptor activity and a carrier, for example, a pharmaceutically acceptable carrier.

This invention still further provides a composition, for example, a pharmaceutical composition, comprising a compound identified by a method according to this invention in an amount effective to decrease mammalian SNORF138 receptor activity and a carrier, for example, a pharmaceutically acceptable carrier.

Furthermore, this invention provides a method of treating an abnormality in a subject wherein the abnormality is alleviated by increasing the activity of a mammalian SNORF138 receptor which comprises administering to the subject a compound which is a mammalian SNORF138 receptor agonist in an amount effective to treat the abnormality.

This invention additionally provides a method of treating an abnormality in a subject wherein the abnormality is alleviated by decreasing the activity of a mammalian SNORF138 receptor which comprises administering to the subject a compound which is a mammalian SNORF138 receptor antagonist in an amount effective to treat the abnormality.

In some embodiments, the abnormality is a regulation of a steroid hormone disorder, an epinephrine release disorder, a gastrointestinal disorder, a cardiovascular disorder, an electrolyte balance disorder, hypertension, diabetes, a respiratory disorder, asthma, a reproductive function disorder, an immune disorder, such as psoriasis, an endocrine disorder, a musculoskeletal disorder, a neuroendocrine disorder, a cognitive disorder, a memory disorder, somatosensory and neurotransmission disorders, metabolic disorders, a motor coordination disorder, a sensory integration disorder, a motor integration disorder, a dopaminergic function disorder, an appetite disorder, such as anorexia or obesity, a sensory transmission disorder, drug addiction, an olfaction disorder, an autonomic nervous system disorder, pain, neuropsychiatric disorders, affective disorder, migraine, circadian disorders, visual disorders, urinary disorders, blood coagulation-related disorders, developmental disorders, or ischemia-reperfusion injury-related diseases.

In one embodiment, the mammalian SNORF138 receptor is a human SNORF138 receptor.

This invention also provides a process for making a composition of matter which specifically binds to a mammalian SNORF138 receptor which comprises identifying a chemical compound using a process in accordance with this invention and then synthesizing the chemical compound or a novel structural and functional analog or homolog thereof.

This invention further provides a process for preparing a composition, for example a pharmaceutical composition which comprises admixing a carrier, for example, a pharmaceutically acceptable carrier, and a therapeutically effective amount of a chemical compound identified by a process in accordance with this invention or a novel structural and functional analog or homolog thereof.

This invention further provides a process for preparing a composition, for example a pharmaceutical composition which comprises identifying a chemical compound by a process in accordance with this invention or a novel structural and functional analog or homolog thereof, recovering the chemical compound free of any receptor, and then admixing a carrier, for example, a pharmaceutically acceptable carrier, and a therapeutically effective amount of the chemical compound.

In one embodiment, the mammalian SNORF138 receptor is a human SNORF138 receptor.

This invention provides a pharmaceutical composition made by combining a therapeutically effective amount of the compound of this invention and a pharmaceutically acceptable carrier.

This invention provides a process for making a pharmaceutical composition comprising combining a therapeutically effective amount of the compound of this invention and a pharmaceutically acceptable carrier.

This invention further provides a pharmaceutical composition comprising a therapeutically effective amount of the compound of the invention and a pharmaceutically acceptable carrier. In one embodiment, the amount of the compound is an amount from about 0.01 mg to about 800 mg.

In another embodiment, the amount of the compound is an amount from about 0.01 mg to about 500 mg. In another embodiment, the amount of the compound is an amount from about 0.01 mg to about 250 mg. In another embodiment, the amount of the compound is an amount from about 0.1 mg to about 60 mg. In another embodiment, the amount of the compound is an amount from about 1 mg to about 20 mg. In a further embodiment, the carrier is a liquid and the composition is a solution. In another embodiment, the carrier is a solid and the composition is a powder or tablet. In a further embodiment, the carrier is a gel and the composition is a capsule or suppository.

In the present invention the term “pharmaceutically acceptable carrier” is any pharmaceutical carrier known to those of ordinary skill in the art as useful in formulating pharmaceutical compositions.

In an embodiment of the present invention, the pharmaceutical carrier may be a liquid and the pharmaceutical composition would be in the form of a solution. In another embodiment, the pharmaceutically acceptable carrier is a solid and the composition is in the form of a powder or tablet. In a further embodiment, the pharmaceutical carrier is a gel and the composition is in the form of a suppository or cream. In a further embodiment the compound may be formulated as a part of a pharmaceutically acceptable transdermal patch. In yet a further embodiment, the compound may be delivered to the subject by means of a spray or inhalant.

A solid carrier can include one or more substances which may also act as endogenous carriers (e.g. nutrient or micronutrient carriers), flavoring agents, lubricants, solubilizers, suspending agents, fillers, glidants, compression aids, binders or tablet-disintegrating agents; it can also be an encapsulating material. In powders, the carrier is a finely divided solid which is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Liquid carriers are used in preparing solutions, suspensions, emulsions, syrups, elixirs and pressurized compositions. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmoregulators. Suitable examples of liquid carriers for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate or isopropyl myristate. Sterile liquid carriers are useful in sterile liquid form compositions for parenteral administration. The liquid carrier for pressurized compositions can be halogenated hydrocarbon or other pharmaceutically acceptable propellent.

Liquid pharmaceutical compositions which are sterile solutions or suspensions can be utilized by for example, intramuscular, intrathecal, epidural, intraperitoneal or subcutaneous injection. Sterile solutions can also be administered intravenously. The compounds may be prepared as a sterile solid composition which may be dissolved or suspended at the time of administration using sterile water, saline, or other appropriate sterile injectable medium. Carriers are intended to include necessary and inert binders, suspending agents, lubricants, flavorants, sweeteners, preservatives, dyes, and coatings.

In the subject invention a “therapeutically effective amount” is any amount of a compound which, when administered to a subject suffering from a disease against which the compounds are effective, causes reduction, remission, or regression of the disease. In the subject application, a “subject” is a vertebrate, a mammal, or a human.

The compound can be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like.

The compound can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

Optimal dosages to be administered may be determined by those skilled in the art, and will vary with the particular compound in use, the strength of the preparation, the mode of administration, and the advancement of the disease condition. Additional factors depending on the particular subject being treated will result in a need to adjust dosages, including subject age, weight, gender, diet, and time of administration.

This invention provides a recombinant nucleic acid comprising a nucleic acid encoding a mammalian SNORF138 receptor, wherein the mammalian receptor-encoding nucleic acid hybridizes under high stringency conditions to a nucleic acid encoding a human SNORF138 receptor. This invention provides a recombinant nucleic acid comprising a nucleic acid encoding a mammalian SNORF138 receptor, wherein the receptor has a sequence identical to the sequence of the human SNORF138 receptor encoded by plasmid MSP70-hSNORF138-f (ATCC Patent Deposit Designation PTA-4725).

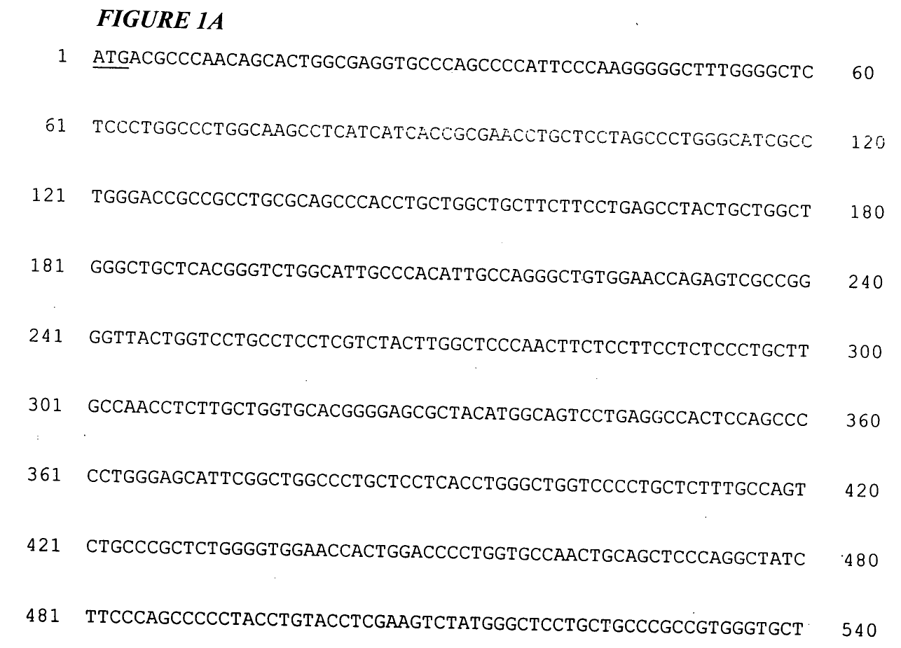

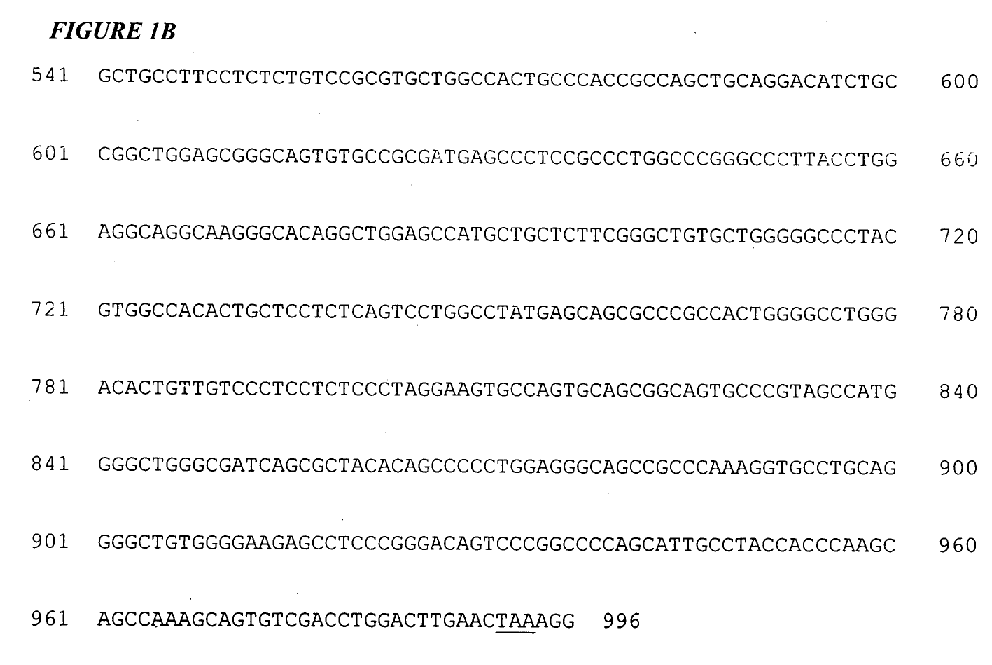

This invention further provides a recombinant nucleic acid comprising a nucleic acid encoding a human SNORF138 receptor, wherein the human SNORF138 receptor comprises an amino acid sequence identical to the sequence of the human SNORF138 receptor as indicated in FIGS. 1A-1B (SEQ ID NO: 1). In one embodiment, the human SNORF138 receptor is encoded by the nucleotide sequence beginning at the start codon at positions 1-3 and ending at the stop codon at positions 991-993 as indicated in FIGS. 1A-1B (SEQ ID NO: 1).

The plasmid MSP70-hSNORF138-f was deposited on Sep. 25, 2002, with the American Type Culture Collection (ATCC), 10801 University Blvd., Manassas, Va. 20110-2209, U.S.A. under the provisions of the Budapest Treaty for the International Recognition of the Deposit of Microorganisms for the Purposes of Patent Procedure and was accorded ATCC Patent Deposit Designation PTA-4725.

This invention also contemplates recombinant nucleic acids which comprise nucleic acids encoding naturally occurring allelic variants of the mammalian SNORF138 receptors described above.

Hybridization methods are well known to those of skill in the art. For purposes of this invention, hybridization under high stringency conditions means hybridization performed at 40° C. in a hybridization buffer containing 50% formamide, 5×SSC, 7 mM Tris, 1× Denhardt's, 25 g/ml salmon sperm DNA; wash at 50° C. in 0.1×SSC, 0.1% SDS.

Throughout this application, the following standard abbreviations are used to indicate specific nucleotide bases:

A=adenine

G=guanine

C=cytosine

T=thymine

M=adenine or cytosine

R=adenine or guanine

W=adenine or thymine

S=cytosine or guanine

Y=cytosine or thymine

K=guanine or thymine

V=adenine, cytosine, or guanine (not thymine)

H=adenine, cytosine, or thymine (not cytosine)

B=cytosine, guanine, or thymine (not adenine)

N=adenine, cytosine, guanine, or thymine (or other modified base such as inosine)

I=inosine

Furthermore, the term “agonist” is used throughout this application to indicate any peptide or non-peptidyl compound which increases the activity of any of the polypeptides of the subject invention. The term “antagonist” is used throughout this application to indicate any peptide or non-peptidyl compound which decreases the activity of any of the polypeptides of the subject invention.

Furthermore, as used herein, the phrase “pharmaceutically acceptable carrier” means any of the standard pharmaceutically acceptable carriers. Examples include, but are not limited to, phosphate buffered saline, physiological saline, water, and emulsions, such as oil/water emulsions.

It is possible that the mammalian SNORF138 receptor gene contains introns and furthermore, the possibility exists that additional introns could exist in coding or non-coding regions. In addition, spliced form(s) of mRNA may encode additional amino acids either upstream of the currently defined starting methionine or within the coding region. Further, the existence and use of alternative exons is possible, whereby the mRNA may encode different amino acids within the region comprising the exon. In addition, single amino acid substitutions may arise via the mechanism of RNA editing such that the amino acid sequence of the expressed protein is different than that encoded by the original gene. (Burns, C. M. et al., 1997; Chu, et al., 1996). Such variants may exhibit pharmacological properties differing from the polypeptide encoded by the original gene.

This invention provides splice variants of the mammalian SNORF138 receptor disclosed herein. This invention further provides for alternate translation initiation sites and alternately spliced or edited variants of nucleic acids encoding the mammalian SNORF138 receptor of this invention.

The nucleic acids of the subject invention also include nucleic acid analogs of the human SNORF138 receptor gene, wherein the human SNORF138 receptor gene comprises the nucleic acid sequence shown in FIGS. 1A-1B (SEQ ID NO: 1) or contained in plasmid MSP70-hSNORF138-f (ATCC Patent Deposit Designation PTA-4725). Nucleic acid analogs of the human SNORF138 receptor genes differ from the human SNORF138 receptor genes described herein in terms of the identity or location of one or more nucleic acid bases (deletion analogs containing less than all of the nucleic acid bases shown in FIGS. 1A-1B or contained in plasmid MSP70-hSNORF138-f, substitution analogs wherein one or more nucleic acid bases shown in FIGS. 1A-1B or contained in plasmid MSP70-hSNORF138-f (ATCC Patent Deposit Designation PTA-4725), are replaced by other nucleic acid bases, and addition analogs, wherein one or more nucleic acid bases are added to a terminal or medial portion of the nucleic acid sequence) and which encode proteins which share some or all of the properties of the proteins encoded by the nucleic acid sequences shown in FIGS. 1A-1B or contained in plasmid MSP70-hSNORF138-f (ATCC Patent Deposit Designation PTA-4725).

In one embodiment of the present invention, the nucleic acid analog encodes a protein which has an amino acid sequence identical to that shown in FIGS. 2A-2B or encoded by the nucleic acid sequence contained in plasmid MSP70-hSNORF138-f (ATCC Patent Deposit Designation PTA-4725). In another embodiment, the nucleic acid analog encodes a protein having an amino acid sequence which differs from the amino acid sequences shown in FIGS. 2A-2B or encoded by the nucleic acid contained in plasmid MSP70-hSNORF138-f (ATCC Patent Deposit Designation PTA-4725). See, for example, PCT International Publication No. WO 01/98330 or Genembl Database Accession No. AC0121016.

In a further embodiment, the protein encoded by the nucleic acid analog has a function which is the same as the function of the receptor proteins having the amino acid sequence shown in FIGS. 2A-2B. In another embodiment, the function of the protein encoded by the nucleic acid analog differs from the function of the receptor protein having the amino acid sequence shown in FIGS. 2A-2B. In another embodiment, the variation in the nucleic acid sequence occurs within the transmembrane (TM) region of the protein. In a further embodiment, the variation in the nucleic acid sequence occurs outside of the TM region.

In another embodiment, the nucleic acid analog encodes a mammalian SNORF138 receptor which has above 75% amino acid identity, preferably above 85% amino acid identity, more preferably above 95% amino acid identity to the SNORF138 receptor encoded by the plasmid MSP70-hSNORF138-f (ATCC Patent Deposit Designation PTA-4725).

This invention provides the above-described isolated nucleic acid, wherein the nucleic acid is DNA. In an embodiment, the DNA is cDNA. In another embodiment, the DNA is genomic DNA. In still another embodiment, the nucleic acid is RNA. Methods for production and manipulation of nucleic acid molecules are well known in the art.

This invention further provides a nucleic acid which is degenerate with respect to the DNA encoding any of the polypeptides described herein. In an embodiment, the nucleic acid comprises a nucleotide sequence which is degenerate with respect to the nucleotide sequence shown in FIGS. 1A-1B (SEQ ID NO: 1) or the nucleotide sequence contained in the plasmid MSP70-hSNORF138-f (ATCC Patent Deposit Designation PTA-4725), that is, a nucleotide sequence which is translated into the same amino acid sequence.

This invention also encompasses DNAs and cDNAs which encode amino acid sequences which differ from those of the polypeptides of this invention, but which should not produce phenotypic changes.

Alternately, this invention also encompasses DNAs, cDNAs, and RNAs which hybridize to the DNA, cDNA, and RNA of the subject invention. Hybridization methods are well known to those of skill in the art.

The nucleic acids of the subject invention also include nucleic acid molecules coding for polypeptide analogs, fragments or derivatives of antigenic polypeptides which differ from naturally-occurring forms in terms of the identity or location of one or more amino acid residues (deletion analogs containing less than all of the residues specified for the protein, substitution analogs wherein one or more residues specified are replaced by other residues and addition analogs wherein one or more amino acid residues is added to a terminal or medial portion of the polypeptides) and which share some or all properties of naturally-occurring forms. These molecules include: the incorporation of codons “preferred” for expression by selected non-mammalian hosts; the provision of sites for cleavage by restriction endonuclease enzymes; and the provision of additional initial, terminal or intermediate DNA sequences that facilitate construction of readily expressed vectors. The creation of polypeptide analogs is well known to those of skill in the art (Spurney, R. F. et al. (1997); Fong, T. M. et al. (1995); Underwood, D. J. et al. (1994); Graziano, M. P. et al. (1996); Guan X. M. et al. (1995)).

The modified polypeptides of this invention may be transfected into cells either transiently or stably using methods well-known in the art, examples of which are disclosed herein. This invention also provides for binding assays using the modified polypeptides, in which the polypeptide is expressed either transiently or in stable cell lines. This invention further provides a compound identified using a modified polypeptide in a binding assay such as the binding assays described herein.

The nucleic acids described and claimed herein are useful for the information which they provide concerning the amino acid sequence of the polypeptide and as products for the large scale synthesis of the polypeptides by a variety of recombinant techniques. The nucleic acid molecule is useful for generating new cloning and expression vectors, transformed and transfected prokaryotic and eukaryotic host cells, and new and useful methods for cultured growth of such host cells capable of expression of the polypeptide and related products.

This invention also provides an isolated nucleic acid encoding species homologs of the SNORF138 receptor encoded by the nucleic acid sequence shown in FIGS. 1A-1B (SEQ ID NO: 1) or encoded by the plasmid MSP70-hSNORF138-f (ATCC Patent Deposit Designation PTA-4725). In one embodiment, the nucleic acid encodes a mammalian SNORF138 receptor homolog which has substantially the same amino acid sequence as does the SNORF138 receptor encoded by the plasmid MSP70-hSNORF138-f (ATCC Patent Deposit Designation PTA-4725).

Examples of methods for isolating and purifying species homologs are described elsewhere (e.g., U.S. Pat. No. 5,602,024, WO94/14957, WO97/26853, WO98/15570).

In another embodiment, the nucleic acid encodes a mammalian SNORF138 receptor homolog which has above 75% amino acid identity to the SNORF138 receptor encoded by the plasmid MSP70-hSNORF138-f (ATCC Patent Deposit Designation PTA-4725); preferably above 85% amino acid identity to the SNORF138 receptor encoded by the plasmid MSP70-hSNORF138-f (ATCC Patent Deposit Designation PTA-4725); most preferably above 95% amino acid identity to the SNORF138 receptor encoded by the plasmid MSP70-hSNORF138-f (ATCC Patent Deposit Designation PTA-4725). In another embodiment, the mammalian SNORF138 receptor homolog has above 70% nucleic acid identity to the SNORF138 receptor gene contained in plasmid MSP70-hSNORF138-f (ATCC Patent Deposit Designation PTA-4725); preferably above 80% nucleic acid identity to the SNORF138 receptor gene contained in the plasmid MSP70-hSNORF138-f (ATCC Patent Deposit Designation PTA-4725); more preferably above 90% nucleic acid identity to the SNORF138 receptor gene contained in the plasmid MSP70-hSNORF138-f (ATCC Patent Deposit Designation PTA-4725).

This invention provides an isolated nucleic acid encoding a mammalian SNORF138 receptor. In one embodiment, the nucleic acid is DNA. In another embodiment, the DNA is cDNA. In another embodiment, the DNA is genomic DNA. In another embodiment, the nucleic acid is RNA.

In one embodiment, the mammalian SNORF138 receptor is a human SNORF138 receptor. In another embodiment, the human SNORF138 receptor has an amino acid sequence identical to that encoded by the plasmid MSP70-hSNORF138-f (ATCC Patent Deposit Designation PTA-4725). In another embodiment, the human SNORF138 receptor has an amino acid sequence identical to the amino acid sequence shown in FIGS. 2A-2B (SEQ ID NO: 2).

This invention provides a purified mammalian SNORF138 receptor protein. In one embodiment, the SNORF138 receptor protein is a human SNORF138 receptor protein.

This invention provides a vector comprising the nucleic acid of this invention. This invention further provides a vector adapted for expression in a cell which comprises the regulatory elements necessary for expression of the nucleic acid in the cell operatively linked to the nucleic acid encoding the receptor so as to permit expression thereof, wherein the cell is a bacterial, amphibian, yeast, insect or mammalian cell. In one embodiment, the vector is a baculovirus. In another embodiment, the vector is a plasmid.

This invention provides a plasmid designated MSP70-hSNORF138-f (ATCC Patent Deposit Designation PTA-4725).

This invention further provides for any vector or plasmid which comprises modified untranslated sequences, which are beneficial for expression in desired host cells or for use in binding or functional assays. For example, a vector or plasmid with untranslated sequences of varying lengths may express differing amounts of the polypeptide depending upon the host cell used. In an embodiment, the vector or plasmid comprises the coding sequence of the polypeptide and the regulatory elements necessary for expression in the host cell.

This invention provides for a cell comprising the vector of this invention. In one embodiment, the cell is a non-mammalian cell. In one embodiment, the non-mammalian cell is a Xenopus oocyte cell or a Xenopus melanophore cell. In another embodiment, the cell is a mammalian cell. In another embodiment, the cell is a COS-7 cell, a human embryonic kidney cell, a HEK293 cell, a NIH-3T3 cell, a LM(tk-) cell, a mouse Y1 cell, or a CHO cell. In another embodiment, the cell is an insect cell. In another embodiment, the insect cell is an Sf9 cell, an Sf21 cell or a Trichoplusia ni 5B-4 cell.

This invention provides a membrane preparation isolated from the cell in accordance with this invention.

Furthermore, this invention provides for a nucleic acid probe comprising at least 15 nucleotides, which probe specifically hybridizes with a nucleic acid encoding a mammalian SNORF138 receptor, wherein the probe has a sequence complementary to a unique sequence present within one of the two strands of the nucleic acid encoding the mammalian SNORF138 receptor contained in plasmid MSP70-hSNORF138-f (ATCC Patent Deposit Designation PTA-4725).

This invention further provides a nucleic acid probe comprising at least 15 nucleotides, which probe specifically hybridizes with a nucleic acid encoding a mammalian SNORF138 receptor, wherein the probe has a sequence complementary to a unique sequence present within (a) the nucleic acid sequence shown in FIGS. 1A-1B (SEQ ID NO: 1) or (b) the reverse complement to (a). In one embodiment, the nucleic acid is DNA. In another embodiment, the nucleic acid is RNA.

As used herein, the phrase “specifically hybridizing” means the ability of a nucleic acid molecule to recognize a nucleic acid sequence complementary to its own and to form double-helical segments through hydrogen bonding between complementary base pairs.

The nucleic acids of this invention may be used as probes to obtain homologous nucleic acids from other species and to detect the existence of nucleic acids having complementary sequences in samples.

The nucleic acids may also be used to express the receptors they encode in transfected cells.

The use of a constitutively active receptor encoded by SNORF138 either occurring naturally without further modification or after appropriate point mutations, deletions or the like, allows screening for antagonists and in vivo use of such antagonists to attribute a role to receptor SNORF138 without prior knowledge of the endogenous ligand.

Use of the nucleic acids further enables elucidation of possible receptor diversity and of the existence of multiple subtypes within a family of receptors of which SNORF138 is a member.

It is contemplated that the receptors of this invention will serve as a valuable tool for designing drugs for treating various pathophysiological conditions such as chronic and acute inflammation, arthritis, autoimmune diseases, transplant rejection, graft vs. host disease, bacterial, fungal, protozoan and viral infections, septicemia, AIDS, pain, psychotic and neurological disorders, including anxiety, depression, schizophrenia, dementia, mental retardation, memory loss, epilepsy, neuromotor disorders, locomotor disorders, respiratory disorders, asthma, eating/body weight disorders including obesity, bulimia, diabetes, anorexia, nausea, hypertension, hypotension, vascular and cardiovascular disorders, ischemia, stroke, cancers, ulcers, urinary retention, sexual/reproductive disorders, circadian rhythm disorders, renal disorders, bone diseases including osteoporosis, benign prostatic hypertrophy, gastrointestinal disorders, nasal congestion, dermatological disorders such as psoriasis, allergies, Parkinson's disease, Alzheimer's disease, acute heart failure, angina disorders, delirium, dyskinesias such as Huntington's disease or Gille's de la Tourette's syndrome, among others and diagnostic assays for such conditions. The receptors of this invention may also serve as a valuable tool for designing drugs for chemoprevention.

Methods of transfecting cells e.g. mammalian cells, with such nucleic acid to obtain cells in which the receptor is expressed on the surface of the cell are well known in the art. (See, for example, U.S. Pat. Nos. 5,053,337; 5,155,218; 5,360,735; 5,472,866; 5,476,782; 5,516,653; 5,545,549; 5,556,753; 5,595,880; 5,602,024; 5,639,652; 5,652,113; 5,661,024; 5,766,879; 5,786,155; and 5,786,157, the disclosures of which are hereby incorporated by reference in their entireties into this application.)

Such transfected cells may also be used to test compounds and screen compound libraries to obtain compounds which bind to the SNORF138 receptor, as well as compounds which activate or inhibit activation of functional responses in such cells, and therefore are likely to do so in vivo. (See, for example, U.S. Pat. Nos. 5,053,337; 5,155,218; 5,360,735; 5,472,866; 5,476,782; 5,516,653; 5,545,549; 5,556,753; 5,595,880; 5,602,024; 5,639,652; 5,652,113; 5,661,024; 5,766,879; 5,786,155; and 5,786,157, the disclosures of which are hereby incorporated by reference in their entireties into this application.)

This invention provides an antibody capable of binding to a mammalian SNORF138 receptor encoded by a nucleic acid encoding a mammalian SNORF138 receptor. In an embodiment of the present invention, the mammalian SNORF138 receptor is a human SNORF138 receptor.

This invention also provides an agent capable of competitively inhibiting the binding of the antibody to a mammalian SNORF138 receptor. In one embodiment, the antibody is a monoclonal antibody or antisera.

Methods of preparing and employing antisense oligonucleotides, antibodies, nucleic acid probes and transgenic animals directed to the SNORF138 receptor are well known in the art. (See, for example, U.S. Pat. Nos. 5,053,337; 5,155,218; 5,360,735; 5,472,866; 5,476,782; 5,516,653; 5,545,549; 5,556,753; 5,595,880; 5,602,024; 5,639,652; 5,652,113; 5,661,024; 5,766,879; 5,786,155; and 5,786,157, the disclosures of which are hereby incorporated by reference in their entireties into this application.)

This invention provides for an antisense oligonucleotide having a sequence capable of specifically hybridizing to RNA encoding a mammalian SNORF138 receptor, so as to prevent translation of such RNA. This invention further provides for an antisense oligonucleotide having a sequence capable of specifically hybridizing to genomic DNA encoding a mammalian SNORF138 receptor, so as to prevent transcription of such genomic DNA. In one embodiment, the oligonucleotide comprises chemically modified nucleotides or nucleotide analogues.

This invention still further provides a pharmaceutical composition comprising (a) an amount of an oligonucleotide in accordance with this invention capable of passing through a cell membrane and effective to reduce expression of a mammalian SNORF138 receptor and (b) a pharmaceutically acceptable carrier capable of passing through the cell membrane.

In one embodiment, the oligonucleotide is coupled to a substance which inactivates mRNA. In another embodiment, the substance which inactivates mRNA is a ribozyme. In another embodiment, the pharmaceutically acceptable carrier comprises a structure which binds to a mammalian SNORF138 receptor on a cell capable of being taken up by the cells after binding to the structure. In another embodiment, the pharmaceutically acceptable carrier is capable of binding to a mammalian SNORF138 receptor which is specific for a selected cell type.

This invention also provides a pharmaceutical composition which comprises an amount of an antibody in accordance with this invention effective to block binding of a ligand to a human SNORF138 receptor and a pharmaceutically acceptable carrier.

This invention further provides a transgenic, nonhuman mammal expressing DNA encoding a mammalian SNORF138 receptor in accordance with this invention. This invention provides a transgenic, nonhuman mammal comprising a homologous recombination knockout of a native mammalian SNORF138 receptor. This invention further provides a transgenic, nonhuman mammal whose genome comprises antisense DNA complementary to DNA encoding a mammalian SNORF138 receptor in accordance with this invention so placed within such genome as to be transcribed into antisense mRNA which is complementary and hybridizes with mRNA encoding the mammalian SNORF138 receptor so as to thereby reduce translation or such mRNA and expression of such receptor. In one embodiment, the DNA encoding the mammalian SNORF138 receptor additionally comprises an inducible promoter. In another embodiment, the DNA encoding the mammalian SNORF138 receptor additionally comprises tissue specific regulatory elements. In another embodiment, the transgenic, nonhuman mammal is a mouse.

This invention also provides a method of detecting expression of a mammalian SNORF138 receptor by detecting the presence of mRNA coding for the mammalian SNORF138 receptor which comprises obtaining total mRNA from the cell and contacting the mRNA so obtained with a nucleic acid probe according to this invention under hybridizing conditions, detecting the presence of mRNA hybridized to the probe, and thereby detecting the expression of the mammalian SNORF138 receptor by the cell.

This invention further provides for a method of detecting the presence of a mammalian SNORF138 receptor on the surface of a cell which comprises contacting the cell with an antibody according to this invention under conditions permitting binding of the antibody to the receptor, detecting the presence of the antibody bound to the cell, and thereby detecting the presence of the mammalian SNORF138 receptor on the surface of the cell.

This invention still further provides a method of determining the physiological effects of varying levels of activity of a mammalian SNORF138 receptor which comprises producing a transgenic, nonhuman mammal in accordance with this invention whose levels of mammalian SNORF138 receptor activity are varied by use of an inducible promoter which regulates mammalian SNORF138 receptor expression.

This invention additionally provides a method of determining the physiological effects of varying levels of activity of a mammalian SNORF138 receptor which comprises producing a panel of transgenic, nonhuman mammals in accordance with this invention each expressing a different amount of a mammalian SNORF138 receptor.

Moreover, this invention provides method for identifying an antagonist capable of alleviating an abnormality wherein the abnormality is alleviated by decreasing the activity of a mammalian SNORF138 receptor comprising administering a compound to a transgenic, nonhuman mammal according to this invention, and determining whether the compound alleviates any physiological and/or behavioral abnormality displayed by the transgenic, nonhuman mammal as a result of overactivity of a mammalian SNORF138 receptor, the alleviation of such an abnormality identifying the compound as an antagonist. In an embodiment, the mammalian SNORF138 receptor is a human SNORF138 receptor.

The invention also provides an antagonist identified by the preceding method according to this invention. This invention further provides a composition, e.g. a pharmaceutical composition comprising an antagonist according to this invention and a carrier, e.g. a pharmaceutically acceptable carrier.

This invention provides a method of treating an abnormality in a subject wherein the abnormality is alleviated by decreasing the activity of a mammalian SNORF138 receptor which comprises administering to the subject an effective amount of the pharmaceutical composition according to this invention so as to thereby treat the abnormality.

In addition, this invention provides a method for identifying an agonist capable of alleviating an abnormality in a subject wherein the abnormality is alleviated by increasing the activity of a mammalian SNORF138 receptor comprising administering a compound to a transgenic, nonhuman mammal according to this invention, and determining whether the compound alleviates any physiological and/or behavioral abnormality displayed by the transgenic, nonhuman mammal, the alleviation of such an abnormality identifying the compound as an agonist. In an embodiment, the mammalian SNORF138 receptor is a human SNORF138 receptor. This invention provides an agonist identified by the preceding method according to this invention. This invention provides a composition, e.g. a pharmaceutical composition comprising an agonist identified by a method according to this invention and a carrier, e.g. a pharmaceutically acceptable carrier.

Moreover, this invention provides a method of treating an abnormality in a subject wherein the abnormality is alleviated by increasing the activity of a mammalian SNORF138 receptor which comprises administering to the subject an effective amount of the pharmaceutical composition of this invention so as to thereby treat the abnormality.

Yet further, this invention provides a method for diagnosing a predisposition to a disorder associated with the activity of a specific mammalian allele which comprises: (a) obtaining DNA of subjects suffering from the disorder; (b) performing a restriction digest of the DNA with a panel of restriction enzymes; (c) electrophoretically separating the resulting DNA fragments on a sizing gel; (d) contacting the resulting gel with a nucleic acid probe capable of specifically hybridizing with a unique sequence included within the sequence of a nucleic acid molecule encoding a mammalian SNORF138 receptor and labeled with a detectable marker; (e) detecting labeled bands which have hybridized to the DNA encoding a mammalian SNORF138 receptor to create a unique band pattern specific to the DNA of subjects suffering from the disorder; (f) repeating steps (a)-(e) with DNA obtained for diagnosis from subjects not yet suffering from the disorder; and (g) comparing the unique band pattern specific to the DNA of subjects suffering from the disorder from step (e) with the band pattern from step (f) for subjects not yet suffering from the disorder so as to determine whether the patterns are the same or different and thereby diagnose predisposition to the disorder if the patterns are the same.

In one embodiment, the disorder is a disorder associated with the activity of a specific mammalian allele is diagnosed.

This invention also provides a method of preparing a purified mammalian SNORF138 receptor according to this invention which comprises: (a) culturing cells which express the mammalian SNORF138 receptor; (b) recovering the mammalian SNORF138 receptor from the cells; and (c) purifying the mammalian SNORF138 receptor so recovered.

This invention further provides a method of preparing a purified mammalian SNORF138 receptor according to this invention which comprises: (a) inserting a nucleic acid encoding the mammalian SNORF138 receptor into a suitable expression vector; (b) introducing the resulting vector into a suitable host cell; (c) placing the resulting host cell in suitable condition permitting the production of the mammalian SNORF138 receptor; (d) recovering the mammalian SNORF138 receptor so produced; and optionally (e) isolating and/or purifying the mammalian SNORF138 receptor so recovered.

Once the gene for a targeted receptor subtype is cloned, it is placed into a recipient cell which then expresses the targeted receptor subtype on its surface. This cell, which expresses a single population of the targeted human receptor subtype, is then propagated resulting in the establishment of a cell line. This cell line, which constitutes a drug discovery system, is used in two different types of assays: binding assays and functional assays. In binding assays, the affinity of a compound for both the receptor subtype that is the target of a particular drug discovery program and other receptor subtypes that could be associated with side effects are measured. These measurements enable one to predict the potency of a compound, as well as the degree of selectivity that the compound has for the targeted receptor subtype over other receptor subtypes. The data obtained from binding assays also enable chemists to design compounds toward or away from one or more of the relevant subtypes, as appropriate, for optimal therapeutic efficacy. In functional assays, the nature of the response of the receptor subtype to the compound is determined. Data from the functional assays show whether the compound is acting to inhibit or enhance the activity of the receptor subtype, thus enabling pharmacologists to evaluate compounds rapidly at their ultimate human receptor subtypes targets permitting chemists to rationally design drugs that will be more effective and have fewer or substantially less severe side effects than existing drugs.

Approaches to designing and synthesizing receptor subtype-selective compounds are well known and include traditional medicinal chemistry and the newer technology of combinatorial chemistry, both of which are supported by computer-assisted molecular modeling. With such approaches, chemists and pharmacologists use their knowledge of the structures of the targeted receptor subtype and compounds determined to bind and/or activate or inhibit activation of the receptor subtype to design and synthesize structures that will have activity at these receptor subtypes.

Combinatorial chemistry involves automated synthesis of a variety of novel compounds by assembling them using different combinations of chemical building blocks. The use of combinatorial chemistry greatly accelerates the process of generating compounds. The resulting arrays of compounds are called libraries and are used to screen for compounds (“lead compounds”) that demonstrate a sufficient level of activity at receptors of interest. Using combinatorial chemistry it is possible to synthesize “focused” libraries of compounds anticipated to be highly biased toward the receptor target of interest.

Once lead compounds are identified, whether through the use of combinatorial chemistry or traditional medicinal chemistry or otherwise, a variety of homologs and analogs are prepared to facilitate an understanding of the relationship between chemical structure and biological or functional activity. These studies define structure activity relationships which are then used to design drugs with improved potency, selectivity and pharmacokinetic properties. Combinatorial chemistry is also used to rapidly generate a variety of structures for lead optimization. Traditional medicinal chemistry, which involves the synthesis of compounds one at a time, is also used for further refinement and to generate compounds not accessible by automated techniques. Once such drugs are defined the production is scaled up using standard chemical manufacturing methodologies utilized throughout the pharmaceutical and chemistry industry.

This invention will be better understood from the Experimental Details which follow. However, one skilled in the art will readily appreciate that the specific methods and results discussed are merely illustrative of the invention as described more fully in the claims which follow thereafter.

Experimental Details

Materials and Methods

Isolation of the Human SNORF138 Receptor

An unannotated putative novel GPCR fragment was discovered in the database of sequences comprising phase I of the human genome project using datamining techniques. PCR primers were designed based on the sequence of this fragment. The PCR primers were subsequently synthesized for the purpose of screening cDNA libraries.

The primers synthesized were:

| | | |
|---|---|---|
| TB1: | 5′- GTTCAAGTCCAGGTCGACACTGC | (SEQ ID NO: 3) |
| TB2: | 5′- GGCACAGGCTGGAGCCATGCTG | (SEQ ID NO: 4) |

A 1375 bp cDNA was isolated from a human kidney cDNA library by PCR screening/sib selection with TB1/TB2. (The human kidney cDNA library was constructed in a plasmid vector.) Sequence analysis revealed a 993 bp open reading frame that could encode a 330 amino acid protein with predicted transmembrane domains and sequence motifs characteristic of the rhodopsin GPCR superfamily. The putative coding region was amplified by PCR using primers KS2588 and KS2589 designed from the cDNA clone and subcloned into a mammalian expression vector. The PCR primers were:

| | | |
|---|---|---|
| KS2588: | 5′-ACGCCCAACAGCACTGGCGAGG | (SEQ ID NO: 5) |
| KS2589: | 5′-CAGCTGAAGGGATCCCCTTTAGTTCAAGTCCAGGTCGAC | (SEQ ID NO: 6) |

The primer KS2589 was designed to incorporate a BamH I restriction enzyme site into the 3′ end of the amplified GPCR coding region. The PCR-amplified fragment was digested with BamH I restriction enzyme prior to ligation into the expression vector MSP 70. The resulting plasmid was designated K1335.

Isolation of Species Homologs of SNORF138 Receptor

A nucleic acid sequence encoding a SNORF138 receptor from other species may be isolated using standard molecular biology techniques and approaches such as those described below:

Approach #1: A genomic library (e.g., cosmid, phage, P1, BAC, YAC) generated from the species of interest may be screened with a ³²P-labeled oligonucleotide probe corresponding to a fragment of the human SNORF138 receptor whose sequence is shown in FIGS. 1A-1B to isolate a genomic clone. The full-length sequence may be obtained by sequencing this genomic clone. If one or more introns are present in the gene, the full-length intronless gene may be obtained from cDNA using standard molecular biology techniques. For example, a forward PCR primer designed in the 5′UT and a reverse PCR primer designed in the 3′UT may be used to amplify a full-length, intronless receptor from cDNA. Standard molecular biology techniques could be used to subclone this gene into a mammalian expression vector.

Approach #2: Standard molecular biology techniques may be used to screen commercial cDNA phage libraries of the species of interest by hybridization under reduced stringency with a ³²P-labeled oligonucleotide probe corresponding to a fragment of the sequences shown in FIGS. 1A-1B. One may isolate a full-length SNORF138 receptor by obtaining a plaque purified clone from the lambda libraries and then subjecting the clone to direct DNA sequencing. Alternatively, standard molecular biology techniques could be used to screen cDNA plasmid libraries by PCR amplification of library pools using primers designed against a partial species homolog sequence. A full-length clone may be isolated by Southern hybridization of colony lifts of positive pools with a ³²P-oligonucleotide probe.

Approach #3: 3′ and 5′ RACE may be utilized to generate PCR products from cDNA derived from the species of interest expressing SNORF138 which contain the additional sequence of SNORF138. These RACE PCR products may then be sequenced to determine the additional sequence. This new sequence is then used to design a forward PCR primer in the 5′UT and a reverse primer in the 3′UT. These primers are then used to amplify a full-length SNORF138 clone from cDNA.

Examples of other species include, but are not limited to, rat, mouse, dog, monkey, hamster and guinea pig.

Host Cells

A broad variety of host cells can be used to study heterologously expressed proteins. These cells include but are not limited to mammalian cell lines such as: COS-7, CHO, LM(tk⁻), HEK293 cells, etc.; insect cell lines such as: Sf9, Sf21, Trichoplusia ni 5B-4, etc.; amphibian cells such as Xenopus oocytes; assorted yeast strains; assorted bacterial cell strains; and others. Culture conditions for each of these cell types is specific and is known to those familiar with the art.

COS-7 cells are grown on 150 mm plates in DMEM with supplements (Dulbecco's Modified Eagle Medium with 10% bovine calf serum, 4 mM glutamine, 100 units/ml penicillin 100 g/ml streptomycin) at 37° C., 5% CO₂. Stock plates of COS-7 cells are trypsinized and split 1:6 every 3-4 days.

HEK cells (Peak^{Rapid} cells™, Edge BioSystems, Gaithersburg, Md.) are maintained in DMEM growth medium supplemented with 10% bovine calf serum, 1% L-glutamine, 50 g/ml gentamycin at 37° C., 5% CO₂.

Transient Expression

DNA encoding proteins to be studied can be transiently expressed in a variety of mammalian, insect, amphibian, yeast, bacterial and other cells lines by several transfection methods including but not limited to; calcium phosphate-mediated, DEAE-dextran mediated; liposomal-mediated, viral-mediated, electroporation-mediated, and microinjection delivery. Each of these methods may require optimization of assorted experimental parameters depending on the DNA, cell line, and the type of assay to be subsequently employed.

A typical protocol for the DEAE-dextran method as applied to COS-7 and HEK293 cells is described as follows. Cells to be used for transfection are split 24 hours prior to the transfection to provide flasks which are 70-80% confluent at the time of transfection. Briefly, 8 g of receptor DNA plus 8 g of any additional DNA needed (e.g. G protein expression vector, reporter construct, antibiotic resistance marker, mock vector, etc.) are added to 9 ml of complete DMEM plus DEAE-dextran mixture (10 mg/ml in PBS). Cells plated into a T225 flask (sub-confluent) are washed once with PBS and the DNA mixture is added to each flask. The cells are allowed to incubate for 30 minutes at 37° C., 5% CO₂. Following the incubation, 36 ml of complete DMEM with 80 M chloroquine is added to each flask and allowed to incubate an additional 3 hours. The medium is then aspirated and 24 ml of complete medium containing 10% DMSO for exactly 2 minutes and then aspirated. The cells are then washed 2 times with PBS and 30 ml of complete DMEM added to each flask. The cells are then allowed to incubate over night. The next day the cells are harvested by trypsinization and reseeded into 96 well plates.

Alternatively, HEK cells may be transfected with the calcium phosphate method according to Jordan, et al. (1996).

Stable Expression

Heterologous DNA can be stably incorporated into host cells, causing the cell to perpetually express a foreign protein. Methods for the delivery of the DNA into the cell are similar to those described above for transient expression but require the co-transfection of an ancillary gene to confer drug resistance on the targeted host cell. The ensuing drug resistance can be exploited to select and maintain cells that have taken up the DNA. An assortment of resistance genes are available including but not restricted to neomycin, kanamycin, and hygromycin. For purposes of studies concerning the receptor of this invention, stable expression of a heterologous receptor protein is typically carrier out in, mammalian cells including but not necessarily restricted to, CHO, HEK293, LM(tk-), etc. In addition native cell lines that naturally carry and express the nucleic acid sequences for the receptor may be used without the need to engineer the receptor complement.

Membrane Preparations

Cell membranes expressing the receptor protein of this invention are useful for certain types of assays including but not restricted to ligand binding assays, GTP— —S binding assays, and others. The specifics of preparing such cell membranes may in some cases be determined by the nature of the ensuing assay but typically involve harvesting whole cells and disrupting the cell pellet by sonication in ice cold buffer (e.g. 20 mM Tris HCl, mM EDTA, pH 7.4 at 4° C.). The resulting crude cell lysate is cleared of cell debris by low speed centrifugation at 200×g for 5 min at 4° C. The cleared supernatant is then centrifuged at 40,000×g for 20 min at 4° C., and the resulting membrane pellet is washed by suspending in ice cold buffer and repeating the high speed centrifugation step. The final washed membrane pellet is resuspended in assay buffer. Protein concentrations are determined by the method of Bradford (1976) using bovine serum albumin as a standard. The membranes may be used immediately or frozen for later use.

Radiolabeled Ligand Binding Assays

Cells expressing the receptor of this invention may be used to screen for ligands for said receptor. The same assays may be used to identify agonists or antagonists of the receptor that may be employed for a variety of therapeutic purposes.

Radioligand binding assays are performed by diluting membranes prepared from cells expressing the receptor in 50 mM Tris buffer (pH=7.4 at 0° C.) containing 0.1% bovine serum albumin (Sigma), aprotinin (0.005 mg/ml, Boehringer Mannheim) and bestatin (0.1 mM, Sigma) as protease inhibitors. The final protein concentration in the assay can be 12-40 g/ml.

Membranes are then incubated with radiolabeled ligand either in the presence or absence of competing ligands on ice for 60 min in a total volume of 250 l in 96 well microtiter plates. The bound ligand is then separated from free ligands by filtration through GF/B filters presoaked in 0.5% polyethyleneimine (PEI), using a Tomtec (Wallac) vacuum filtration device. After addition of Ready Safe (Beckman) scintillation fluid, bound radioactivity is quantitated using a Trilux (Wallac) scintillation counter (approximately 40% counting efficiency of bound counts). Alternatively, it may be preferable to collect bound ligand and then separate ligand from receptor using procedures well known in the art. Data is fit to non-linear curves using GraphPad Prism.

In this manner, agonist or antagonist compounds that bind to the receptor may be identified as they inhibit the binding of the radiolabeled ligand to the membrane protein of cells expressing the said receptor. Non-specific binding is defined as the amount of radioactivity remaining after incubation of membrane protein in the presence of 100 nM of the unlabeled peptide corresponding to the radioligand used. In equilibrium saturation binding assays membrane preparations or intact cells transfected with the receptor are incubated in the presence of increasing concentrations of the labeled compound to determine the binding affinity of the labeled ligand. The binding affinities of unlabeled compounds may be determined in equilibrium competition binding assays, using a fixed concentration of labeled compound in the presence of varying concentrations of the displacing ligands.

Functional Assays

Cells expressing the receptor DNA of this invention may be used to screen for ligands to said receptor using functional assays. Once a ligand is identified the same assays may be used to identify agonists or antagonists of the receptor that may be employed for a variety of therapeutic purposes. It is well known to those in the art that the over-expression of a G-protein coupled receptor can result in the constitutive activation of intracellular signaling pathways. In the same manner, over-expression of the receptors of the present invention in any cell line as described above, can result in the activation of the functional responses described below, and any of the assays herein described can be used to screen for agonist, partial agonist, inverse agonist and antagonist ligands of the SNORF138 receptor.

A wide spectrum of assays can be employed to screen for the presence of receptor SNORF138 ligands. These assays range from traditional measurements of total inositol phosphate accumulation, cAMP levels, intracellular calcium mobilization, and potassium currents, for example; to systems measuring these same second messengers but which have been modified or adapted to be of higher throughput, more generic and more sensitive; to cell based assays reporting more general cellular events resulting from receptor activation such as metabolic changes, differentiation, cell division/proliferation. Description of several such assays follow.

Cyclic AMP (cAMP) Assay

For the purpose of this invention, human embryonic kidney cells (Peak^{Rapid} cells™, Edge BioSystems, Gaithersburg, Md.) were trypsinized and transfected with human SNORF138 and G z DNA (16 and 4 g per T150 flask, respectively) using the calcium-phosphate transfection method (Jordan, et al, 1996). Mock transfection was also performed with vector DNA plus G z DNA for comparison. Five hours after plating the cells, the medium was removed and replaced with fresh medium. The following day, the cells were trypsinized and seeded onto poly D-lysine coated Costar 96-well plates (55,000 cells/well), and incubated overnight. 48 hours after transfection, the receptor-mediated stimulation or inhibition of cyclic AMP (cAMP) formation is assayed in an HBS (HEPES-buffered saline) buffer containing: HEPES (20 mM), NaCl (150 mM), CaCl₂ (1 mM), MgCl₂ (1 mM), KCl (5 mM) glucose (10 mM), BSA (0.2%), theophylline (0.9 gm/liter), pH 7.4. Cells are washed twice with assay buffer and are incubated in the same buffer for 20 min at 37° C., in 5% CO₂. For cAMP stimulation assays, test compounds are added and cells are incubated for 10 min at 37° C. For cAMP inhibition assays, test compounds are added and cells are incubated for 5 min at 37° C., after which forskolin (10 M) is added to each well and cells are incubated for additional 5 min at 37° C. The medium is then decanted and the reaction stopped by the addition of 100 mM HCl. The plates are stored at −20° C. for 2-5 days. For cAMP measurement, plates are thawed and the cAMP content in each well is measured by cAMP Scintillation Proximity Assay (Amersham Pharmacia Biotech). Radioactivity is quantified using microbeta Trilux counter (Wallac).

Alternatively, COS-7 cells can be transiently transfected with the receptor gene using the DEAE-dextran method and plated in 96-well plates. 48 hours after transfection, cells are washed twice with Dulbecco's phosphate buffered saline (PBS) supplemented with 10 mM HEPES, 10 mM glucose and 5 mM theophylline and are incubated in the same buffer for 20 min at 37° C., in 5% CO₂. Test compounds are added and cells are incubated for an additional 10 min at 37° C. The medium is then aspirated and the reaction stopped by the addition of 100 mM HCl. The plates are stored at −20° C. for 2-5 days. For cAMP measurement, plates are thawed and the cAMP content in each well is measured by cAMP Scintillation Proximity Assay (Amersham Pharmacia Biotech). Radioactivity is quantified using microbeta Trilux counter (Wallac).

Arachidonic Acid Release Assay

Cells expressing the receptor are seeded into 96 well plates or other vessels and grown for 3 days in medium with supplements. ³H-arachidonic acid (specific activity=0.75 Ci/ml) is delivered as a 100 L aliquot to each well and samples are incubated at 37° C., 5% CO₂ for 18 hours. The labeled cells are washed three times with medium. The wells are then filled with medium and the assay is initiated with the addition of test compounds or buffer in a total volume of 250 L. Cells are incubated for 30 min at 37° C., 5% CO₂. Supernatants are transferred to a microtiter plate and evaporated to dryness at 75° C. in a vacuum oven. Samples are then dissolved and resuspended in 25 L distilled water. Scintillant (300 L) is added to each well and samples are counted for ³H in a Trilux plate reader. Data are analyzed using nonlinear regression and statistical techniques available in the GraphPAD Prism package (San Diego, Calif.).

Intracellular Calcium Mobilization Assays

The intracellular free calcium (Ca²⁺) concentration may be measured by microspectrofluorimetry using the fluorescent indicator dye Fura-2/AM (Bush et al., 1991). Cells expressing the receptor are seeded onto a 35 mm culture dish containing a glass coverslip insert and allowed to adhere overnight. Cells are then washed with HBS and loaded with 100 L of Fura-2/AM (10 M) for 20 to 40 min. After washing with HBS to remove the Fura-2/AM solution, cells are equilibrated in HBS for 10 to 20 min. Cells are then visualized under the 40× objective of a Leitz Fluovert FS microscope and fluorescence emission is determined at 510 nM with excitation wavelengths alternating between 340 nM and 380 nM. Raw fluorescence data are converted to Ca²⁺ concentrations using standard Ca²⁺ concentration curves and software analysis techniques.

In another method, the measurement of intracellular Ca²⁺ can also be performed on a 96-well (or higher) format and with alternative Ca²⁺-sensitive indicators, preferred examples of these are: aequorin, Fluo-3, Fluo-4, Fluo-5, Calcium Green-1, Oregon Green, and 488 BAPTA. After activation of the receptors with agonist ligands the emission elicited by the change of intracellular Ca 2+concentration can be measured by a luminometer, or a fluorescence imager; a preferred example of this is the fluorescence imager plate reader (FLIPR™, Molecular Devices).

Cells expressing the receptor of interest are plated into clear, flat-bottom, black-walled 96-well plates (Costar) at a density of 80,000-150,000 cells per well and allowed to incubate for 48 hr at 5% CO₂, 37° C. The growth medium is aspirated and 100 L of loading medium containing Fluo-3 dye is added to each well. The loading medium contains: 20 mM HEPES (Sigma), 0.1% BSA (Sigma), dye/pluronic acid mixture (e.g. 1 mM Fluo-3/AM (Molecular Probes) and 10% pluronic acid (Molecular Probes) mixed immediately before use), and 2.5 mM probenecid (Sigma) (prepared fresh). The cells are allowed to incubate for about 1 hour at 5% CO₂, 37° C.

The compounds of interest are diluted in wash buffer (Hank's BSS (without phenol red), 20 mM HEPES, 2.5 mM probenecid) to a 4×final concentration and aliquoted into a clear v-bottom plate (Nunc). Following the dye incubation, the cells are washed 4 times to remove excess dye using a Denley plate washer. 100 L final volume of wash buffer is then added to each cell well. Compounds are added to the cell plates and responses are measured using the FLIPR™ instrument. The data are then collected and analyzed using the FLIPR™ software and Graphpad Prism.

Antagonist ligands are identified by the inhibition of the signal elicited by agonist ligands.

GTP S Functional Assay

Membranes from cells expressing the receptor are suspended in assay buffer (e.g., 50 mM Tris, 100 mM NaCl, 5 mM MgCl₂, 10 M GDP, pH 7.4) with or without protease inhibitors (e.g., 0.1% bacitracin). Membranes are incubated on ice for 20 minutes, transferred to a 96-well Millipore microtiter GF/C filter plate and mixed with GTP ³⁵S (e.g., 250,000 cpm/sample, specific activity −1000 Ci/mmol) plus or minus unlabeled GTP S (final concentration=100 M). Final membrane protein concentration ˜90 g/ml. Samples are incubated in the presence or absence of test compounds for 30 min. at room temperature, then filtered on a Millipore vacuum manifold and washed three times with cold (4° C.) assay buffer. Samples collected in the filter plate are treated with scintillant and counted for ³⁵S in a Trilux (Wallac) liquid scintillation counter. It is expected that optimal results are obtained when the receptor membrane preparation is derived from an appropriately engineered heterologous expression system, i.e., an expression system resulting in high levels of expression of the receptor and/or expressing G-proteins having high turnover rates (for the exchange of GDP for GTP). GTP S assays are well-known to those skilled in the art, and it is contemplated that variations on the method described above, such as are described by Tian et al. (1994) or Lazareno and Birdsall (1993), may be used.

Microphysiometric Assay

Because cellular metabolism is intricately involved in a broad range of cellular events (including receptor activation of multiple messenger pathways), the use of microphysiometric measurements of cell metabolism can in principle provide a generic assay of cellular activity arising from the activation of any orphan receptor regardless of the specifics of the receptor's signaling pathway.

General guidelines for transient receptor expression, cell preparation and microphysiometric recording are described elsewhere (Salon, J. A. and Owicki, J. A., 1996). Typically cells expressing receptors are harvested and seeded at 3×10⁵ cells per microphysiometer capsule in complete media 24 hours prior to an experiment. The media is replaced with serum free media 16 hours prior to recording to minimize non-specific metabolic stimulation by assorted and ill-defined serum factors. On the day of the experiment the cell capsules are transferred to the microphysiometer and allowed to equilibrate in recording media (low buffer RPMI 1640, no bicarbonate, no serum (Molecular Devices Corporation, Sunnyvale, Calif.) containing 0.1% fatty acid free BSA), during which a baseline measurement of basal metabolic activity is established.

A standard recording protocol specifies a 100 l/min flow rate, with a 2 min total pump cycle which includes a 30 sec flow interruption during which the acidification rate measurement is taken. Ligand challenges involve a 1 min 20 sec exposure to the sample just prior to the first post challenge rate measurement being taken, followed by two additional pump cycles for a total of 5 min 20 sec sample exposure. Typically, drugs in a primary screen are presented to the cells at 10 M final concentration.

Follow up experiments to examine dose-dependency of active compounds are then done by sequentially challenging the cells with a drug concentration range that exceeds the amount needed to generate responses ranging from threshold to maximal levels. Ligand samples are then washed out and the acidification rates reported are expressed as a percentage increase of the peak response over the baseline rate observed just prior to challenge.

MAP Kinase Assay

MAP kinase (mitogen activated kinase) may be monitored to evaluate receptor activation. MAP kinase is activated by multiple pathways in the cell. A primary mode of activation involves the ras/raf/MEK/MAP kinase pathway. Growth factor (tyrosine kinase) receptors feed into this pathway via SHC/Grb-2/SOS/ras. Gi coupled receptors are also known to activate ras and subsequently produce an activation of MAP kinase. Receptors that activate phospholipase C (such as Gq/G11-coupled) produce diacylglycerol (DAG) as a consequence of phosphatidyl inositol hydrolysis. DAG activates protein kinase C which in turn phosphorylates MAP kinase.

MAP kinase activation can be detected by several approaches. One approach is based on an evaluation of the phosphorylation state, either unphosphorylated (inactive) or phosphorylated (active). The phosphorylated protein has a slower mobility in SDS-PAGE and can therefore be compared with the unstimulated protein using Western blotting. Alternatively, antibodies specific for the phosphorylated protein are available (New England Biolabs) which can be used to detect an increase in the phosphorylated kinase. In either method, cells are stimulated with the test compound and then extracted with Laemmli buffer. The soluble fraction is applied to an SDS-PAGE gel and proteins are transferred electrophoretically to nitrocellulose or Immobilon. Immunoreactive bands are detected by standard Western blotting technique. Visible or chemiluminescent signals are recorded on film and may be quantified by densitometry.

Another approach is based on evaluation of the MAP kinase activity via a phosphorylation assay. Cells are stimulated with the test compound and a soluble extract is prepared. The extract is incubated at 30° C. for 10 min with gamma-³²P-ATP, an ATP regenerating system, and a specific substrate for MAP kinase such as phosphorylated heat and acid stable protein regulated by insulin, or PHAS-I. The reaction is terminated by the addition of H₃PO₄ and samples are transferred to ice. An aliquot is spotted onto Whatman P81 chromatography paper, which retains the phosphorylated protein. The chromatography paper is washed and counted for ³²P in a liquid scintillation counter. Alternatively, the cell extract is incubated with gamma-³²P-ATP, an ATP regenerating system, and biotinylated myelin basic protein bound by streptavidin to a filter support. The myelin basic protein is a substrate for activated MAP kinase. The phosphorylation reaction is carried out for 10 min at 30° C. The extract can then by aspirated through the filter, which retains the phosphorylated myelin basic protein. The filter is washed and counted for ³²P by liquid scintillation counting.

Cell Proliferation Assay

Receptor activation of the orphan receptor may lead to a mitogenic or proliferative response which can be monitored via ³H-thymidine uptake. When cultured cells are incubated with ³H-thymidine, the thymidine translocates into the nuclei where it is phosphorylated to thymidine triphosphate. The nucleotide triphosphate is then incorporated into the cellular DNA at a rate that is proportional to the rate of cell growth. Typically, cells are grown in culture for 1-3 days. Cells are forced into quiescence by the removal of serum for 24 hrs. A mitogenic agent is then added to the media. 24 hrs later, the cells are incubated with ³H-thymidine at specific activities ranging from 1 to 10 Ci/ml for 2-6 hrs. Harvesting procedures may involve trypsinization and trapping of cells by filtration over GF/C filters with or without a prior incubation in TCA to extract soluble thymidine. The filters are processed with scintillant and counted for ³H by liquid scintillation counting. Alternatively, adherent cells are fixed in MeOH or TCA, washed in water, and solubilized in 0.05% deoxycholate/0.1 N NaOH. The soluble extract is transferred to scintillation vials and counted for ³H by liquid scintillation counting.

Alternatively, cell proliferation can be assayed by measuring the expression of an endogenous or heterologous gene product, expressed by the cell line used to transfect the orphan receptor, which can be detected by methods such as, but not limited to, florescence intensity, enzymatic activity, immunoreactivity, DNA hybridization, polymerase chain reaction, etc.

Promiscuous Second Messenger Assays

It is not possible to predict, a priori and based solely upon the GPCR sequence, which of the cell's many different signaling pathways any given receptor will naturally use. It is possible, however, to coax receptors of different functional classes to signal through a pre-selected pathway through the use of promiscuous G subunits. For example, by providing a cell based receptor assay system with an endogenously supplied promiscuous G subunit such as G ₁₅ or G ₁₆ or a chimeric G subunit such as G _{qz}, a GPCR, which might normally prefer to couple through a specific signaling pathway (e.g., Gₛ, Gᵢ, G_{q}, G₀, etc.), can be made to couple through the pathway defined by the promiscuous G subunit and upon agonist activation produce the second messenger associated with that subunit's pathway. In the case of G ₁₅, G ₁₆ and/or G _{qz} this would involve activation of the G_{q} pathway and production of the second messenger IP₃. Through the use of similar strategies and tools, it is possible to bias receptor signaling through pathways producing other second messengers such as Ca⁺⁺, cAMP, and K⁺ currents, for example (Milligan and Rees, 1999).

It follows that the promiscuous interaction of the exogenously supplied G subunit with the receptor alleviates the need to carry out a different assay for each possible signaling pathway and increases the chances of detecting a functional signal upon receptor activation.

Methods for Recording Currents in Xenopus oocytes

In the present invention, oocytes are harvested from Xenopus laevis as previously described (Quick and Lester, 1994; Smith et al., 1997). Oocytes were injected with a cocktail of T3-driven hSN138 cDNA, T3-RNA polymerase and dNTP with or without T3-driven CFTR cDNA directly into the cytoplasm as previously described (Geib et al., 2001). After 2 days of incubation at 15° C., dual electrode voltage clamp was performed using 3 M KCl-filled glass microelectrodes with resistances of 1-2 MΩ. Unless otherwise specified, oocytes were voltage-clamped at a holding potential of −80 mV. During recordings, oocytes were bathed in continuously flowing (1-3 ml/min) medium containing 96 mM NaCl, 2 mM KCl, 1.8 mM CaCl₂, and 5 mM HEPES, pH 7.5 (ND96 Buffer). Drugs were applied by local perfusion from a 10 ml glass capillary tube at a distance of 0.5 mm from the oocyte.

Heterologous expression of GPCRs in Xenopus oocytes has been widely used to determine the identity of signaling pathways activated by agonist stimulation. CFTR channel activity is heavily regulated by the cytosolic levels of cyclic AMP which can be increased by activation of G_{S}-coupled adenylyl cyclase (AC). Therefore, activation of G_{S}-coupled AC pathway is assayed by applying test compound in ND96 solution to oocytes and observing the changes of inward CFTR currents at a holding potential of −80 mV.

Alternatively, activation of the phospholipase C (PLC) pathway is assayed by applying a test compound in ND96 solution to oocytes previously injected with mRNA or cDNA for the human SNORF138 and observing inward currents at a holding potential of approximately −80 mV. The appearance of currents that reverse at −25 mV and display other properties of the Ca⁺⁺-activated Cl⁻ channel is indicative of receptor-acitivation of PLC and release of IP₃ and intracellular Ca⁺⁺. Such activation is exhibited by GPCRs that couple to G_{q} or G₁₁.

Measurement of inwardly rectifying K⁺ (potassium) channel (GIRK) activity may be monitored in oocytes that have been co-injected with mNA or cDNA encoding the mammalian receptor plus GIRK subunits. GIRK gene products co-assemble to form a G-protein activated potassium channel known to be activated (i.e., stimulated) by a number of GPCRs that couple to Gᵢ or Gₒ (Kubo et al., 1993; Dascal et al. 1993) Oocytes expressing the mammalian receptor plus the GIRK subunits are tested for test compound responsitivity by measuring K⁺ currents in elevated K⁺ solution containing 49 mM K⁺.

Inositol Phosphate Assay

Human SNORF138 receptor-mediated activation of the inositol phosphate (IP) second messenger pathways may be assessed by radiometric measurement of IP products.

For example, in a 96 well microplate format assay, COS-7 cells expressing the receptor of interest are plated at a density of 70,000 cells per well and allowed to incubate for 24 hours. The cells are then labeled with 0.5 Ci [³H]myo-inositol overnight at 37° C., 5% CO₂. Immediately before the assay, the medium is removed and replaced with 180 L of Phosphate-Buffered Saline (PBS) containing 10 mM LiCl. The plates are then incubated for 20 min at 37° C., 5% CO₂. Following the incubation, the cells are challenged with agonist (20 l/well; 10×concentration) for 30 min at 37° C. The challenge is terminated by the addition of 100 L of 5% v/v trichloroacetic acid, followed by incubation at 4° C. for greater than 30 minutes. Total IPs are isolated from the lysate by ion exchange chromatography. Briefly, the lysed contents of the wells are transferred to a Multiscreen HV filter plate (Millipore) containing Dowex AG1-X8 (200-400 mesh, formate form). The filter plates are prepared by adding 100 L of Dowex AG1-X8 suspension (50% v/v, water: resin) to each well. The filter plates are placed on a vacuum manifold to wash or elute the resin bed. Each well is first washed 2 times with 200 l of 5 mM myo-inositol. Total [³H]inositol phosphates are eluted with 75 l of 1.2M ammonium formate/0.1M formic acid solution into 96-well plates. 200 L of scintillation cocktail is added to each well, and the radioactivity is determined by liquid scintillation counting.

Generation of Baculovirus

The coding region of DNA encoding the human receptor disclosed herein may be subcloned into pBlueBacIII into existing restriction sites or sites engineered into sequences 5′ and 3′ to the coding region of the polypeptides. To generate baculovirus, 0.5 g of viral DNA (BaculoGold) and 3 g of DNA construct encoding a polypeptide may be co-transfected into 2×10⁶ Spodoptera frugiperda insect Sf9 cells by the calcium phosphate co-precipitation method, as outlined by Pharmingen (in “Baculovirus Expression Vector System: Procedures and Methods Manual”). The cells then are incubated for 5 days at 27° C.

The supernatant of the co-transfection plate may be collected by centrifugation and the recombinant virus plaque purified. The procedure to infect cells with virus, to prepare stocks of virus and to titer the virus stocks are as described in Pharmingen's manual.

Localization of RNA Coding for Human SNORF138 Receptor

Quantitative PCR using a fluorogenic probe with real time detection: Quantitative PCR using fluorogenic probes used to characterize the distribution of SNORF138 RNA. This assay utilizes two oligonucleotides for conventional PCR amplification and a third specific oligonucleotide probe that is labeled with a reporter at the 5′ end and a quencher at the 3′ end of the oligonucleotide. In the instant invention, FAM (6-carboxyfluorescein) was used as the reporter, and Black Hole Quencher-1™ (BH1) (Biosearch) was used as the quencher. As amplification progresses, the labeled oligonucleotide probe hybridizes to the gene sequence between the two oligonucleotides used for amplification. The nuclease activity of Taq thermostable DNA polymerase is utilized to cleave the labeled probe. This separates the quencher from the reporter and generates a fluorescent signal that is directly proportional to the amount of amplicon generated. This labeled probe confers a high degree of specificity. Non-specific amplification is not detected as the labeled probe does not hybridize and as a consequence is not cleaved. All experiments were conducted in a PE7700 Sequence Detection System (PE Biosystems, Foster City, Calif.).

Quantitative RT-PCR: Quantitative RT-PCR was used for the detection of SNORF138 RNA.

For use as a template in quantitative PCR reactions, cDNA was synthesized by reverse transcription from total human RNA. Reverse transcription by SuperScriptII RNAse H⁻ and (GibcoBRL/life Technologies) was primed using random hexamers. Parallel reactions included ³²P labeled dCTP to allow quantification of the cDNA. Following reverse transcription, cDNA was phenol/chloroform extracted and precipitated. Incorporation of ³²P dCTP was assessed after precipitation with trichloroacetic acid and the amount of cDNA synthesized was calculated.

Primers were designed to amplify a region of human SNORF138. Primers with the following sequences were used:

| | |
|---|---|
| Forward primer: | |
| SNORF138-343F | |
| 5′-GCAGAGGCCCTTCCTTTAGTTC -3′ | (SEQ ID NO: 7) |
| Reverse primer: | |
| SNORF138-414R | |
| 5′-CCCAGCATTGCCTACCACC -3′ | (SEQ ID NO: 8) |
| Fluorogenic oligonucleotide probe: | |
| SNORF138 -370T | |
| 5′(6-FAM)-CAGGTCGACACTGCTTTGGCTGCTT -(BHQ-1)3′ | (SEQ ID NO: 9) |

Using these primer pairs, amplicon length is 72 bp for human SNORF138. Each PCR reaction contained 3.0 ng cDNA. Oligonucleotide concentrations were: 500 nM of forward and reverse primers, and 200 nM of fluorogenic probe. PCR reactions were carried out in 50 ml volumes using TaqMan universal PCR master mix (PE Applied Biosystems). Buffer for RT-PCR reactions contained a fluor used as a passive reference (ROX: Perkin Elmer proprietary passive reference I). All reagents for PCR (except cDNA and oligonucleotide primers) were obtained from Perkin Elmer (Foster City, Calif.). Reactions were carried in a PE7700 sequence detection system (PE Applied Biosystems) using the following thermal cycler profile: 50EC 2 min., 95EC 10 min., followed by 40 cycles of: 95EC, 15 sec., 60EC 1 min.

Standard curves for quantification of human SNORF138 were constructed using genomic DNA. Negative controls consisted of mRNA blanks, as well as primer and mRNA blanks. To confirm that the mRNA was not contaminated with genomic DNA, PCR reactions were carried out without reverse transcription using Taq DNA polymerase. Integrity of RNA was assessed by amplification of RNA coding for cyclophilin or glyceraldehyde 3-phosphate dehydrogenase (GAPDH). Following reverse transcription and PCR amplification, data was analyzed using PE Biosystems sequence detection software. The fluorescent signal from each well was normalized using an internal passive reference, and data was fitted a standard curve to obtain relative quantities of SNORF138 expression.

Localization of Human SNORF138 in Diabetic Tissue:

To determine if the level of SNORF 138 transcripts is altered in diabetes, quantitative PCR was performed on cDNA synthesized from total RNA extracted from human skeletal muscle and peri-renal adipose tissue. Tissue samples were obtained from diabetic donors that were insulin dependant for a minimum of five years. Control samples were obtained from normal donors (perirenal adipose), or purchased (skeletal muscle; Clonetech). Tissue was homogenized in TRIZOL (Invitrogen), extracted with chloroform, alcohol precipitated and quantified. To assess integrity of the isolated RNA, a small fraction of the RNA was size fractionated using an Agilent BioAnalyzer. Contaminating DNA was removed from the samples using DNAse. Reverse transcription was carried out using SuperScript II (Invitrogen). PCR reactions were carried out in 50 l volumes using Taq DNA polymerase (Perkin Elmer). The primer, probe and PCR conditions used were the same as previously described.

Each human SNORF138 PCR reaction contained cDNA from either a diabetic or normal donor pool. Expression levels assayed in cDNA from individual diseased donors was compared to cDNA from a pool of normal donors. For perirenal adipose cDNA, the normal pool contained cDNA synthesized two donors, and skeletal muscle pool contained cDNA from eight donors. To equalize the amount of diabetic and normal cDNA in each reaction the amounts of cDNA normalized to cyclophilin. Each cDNA was assayed for cyclophilin in increasing quantities ranging from an estimated 0.25 ng to 3.0 ng. The cycle threshold values of the diabetic samples were compared to those of pooled normal samples. The amount of diabetic cDNA that had the same cycle threshold as the normal cDNA was then used in the quantitative PCR assay. Oligonucleotide concentrations were: 500 nM of forward and reverse primers, and 50 nM of fluorogenic probe. The amplification reagent in each reaction was 1×TaqMan Universal PCR Master Mix which contains AmpliTaq Gold DNA Polymerase, AmpErase UNG, dNTPS with dUTP, passive reference, and optimized buffer components (Perkin Elmer proprietary enzymes and buffers).

Following PCR amplification, data was analyzed using Perkin Elmer sequence detection software. The fluorescent signal from each well was normalized using an internal passive reference, and data from diabetic tissue was compared to the normal. Data was again normalized to a measurement of cyclophilin in each assay plate.

Chemical Compounds

The IUPAC chemical name for Betamethasone Valerate is fluoro-11b,17,21-trihydroxy-16b-methylpregna-1,4-diene-3,20-dione 17-valerate; registry number: 2152-44-5. Betamethasone Valerate can be purchased for research use as a white, crystallized powder from Pharmacia Fine Chemicals or from a number of other commercial vendors. The structure for Betamethasone Valerate is as follows:

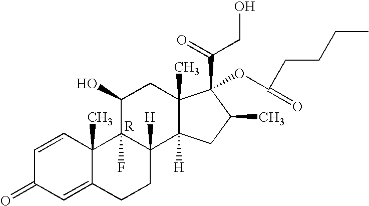

Betamethasone Valerate is classified under the corticosteroid chemical class and has known therapeutic uses as an anti-inflammatory and/or immunosuppressive agent.

Results

Isolation of a Full-Length Human SNORF138 Receptor

The full-length coding sequence of human SNORF138 receptor was isolated from a human kidney cDNA library. PCR primers used to screen the cDNA library were designed based on the sequence of an unannotated putative novel GPCR fragment found in the human genome database using datamining techniques. The resultant putative full-length hSNORF138 sequence consists of a 993 bp nucleotide coding region which can encode a 330 amino acid protein.

The predicted hSNORF138 amino acid sequence contains seven putative transmembrane domains and sequence motifs characteristic of the rhodopsin GPCR superfamily. Searches of sequence databases using BLAST analysis (GCG Wisconsin Package Version 10.3, Accelrys Inc., San Diego, Calif.) show that the most closely related human sequences are GPCRs with the following overall amino acid identities: gpr27 orphan receptor, 36%; edg-8 (sphingosine-1-phosphate receptor), 33%; beta3 adrenergic receptor, 33%.

Activation of CFTR Currents in hSN138-Expressing Xenopus oocytes

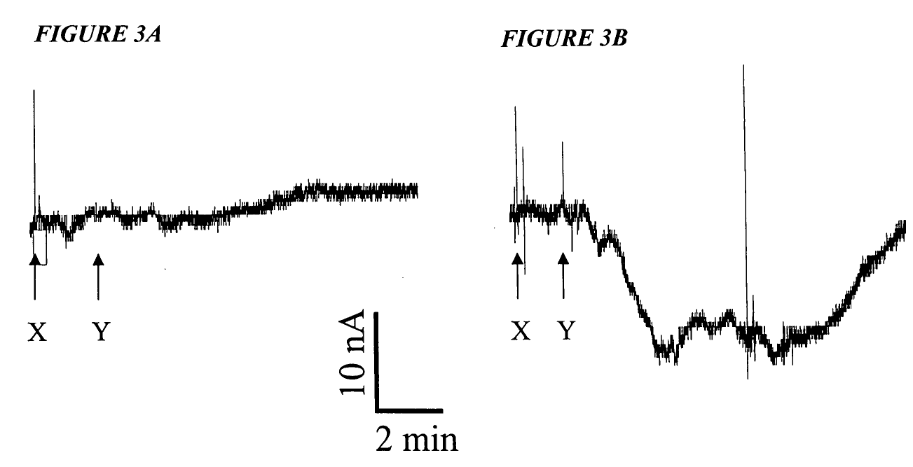

In Xenopus oocytes expressing CFTR alone, no significant changes in CFTR currents were observed upon a bullet application of 50 M betamethasone valerate (n=4, see FIG. 3). However, 50 M betamethasone valerate elicited a significant increase of CFTR currents (9.2±1.9 nA, n=8) in oocytes expressing both hSN138 and CFTR (see FIG. 3) indicating that the glucocorticosteroid betamethasone valerate stimulates Gs-coupled AC pathway via activation of hSN138, which in turn leads to an increase of CFTR currents.

Detection of mRNA coding for human SNORF138 receptor: mRNA was isolated from multiple tissues (Table 1) and assayed as described.

Expression profiling using a broad tissue panel indicates that transcripts for this receptor are expressed in relatively low amounts in all tissues assayed. None of the 31 tissues assayed showed very high expression levels. The highest levels of SNORF138 transcripts were found in placenta and spleen. The presence of relatively high expression in the placenta, combine with the significant levels detected in the uterus suggest that SNORF138 may play a role in gestational disorders. The relatively high levels detected in the spleen suggest a role in modulating the immune system.

Expressing at a somewhat lower level are adipose tissue, kidney medulla, large intestine and lung. Moderate expression levels are detected in three subtypes of adipose tissue: subcutaneous, peri-renal, and omental. Although absolute expression levels are not high as compared to other GPCRs (in adipose tissue or elsewhere), the expression levels in adipose tissues are fairly high when compared to the expression of SNORF138 in other tissues. This is important because of the potential role that adipose tissue, especially omental and peri-renal has in metabolic disorders, as well as obesity and lipid metabolism. The moderate levels of SNORF138 detected in the lung imply a role in modulation respiratory function, or perhaps in the regulation of the smooth muscle surrounding the airways. All regions of the gastrointestinal tract (GI) that were assayed express SNORF138 RNA. This suggests a role in modulation gastric motility of other aspects of GI function. Expression in the kidney is notable. Despite the fact that expression levels in the kidney are relatively low, SNORF138 is one of the few genes examined that has a differential expression pattern in the kidney cortex vs. medulla. Most GPCRs assayed by our group have equivalent expression levels in both kidney cortex and medulla. Expression levels of SNORF138 are 4-5 fold higher in the medulla than in the cortex. This suggests that SNORF138 may play a role in regulation of water reabsorbtion or other aspects of renal homeostasis.

In addition to the potential therapeutic applications identified in Table 1, the localization data for RNA encoding the human SNORF138 receptor indicates that the DNA encoding the human SNORF138 receptor can be used to predict the likelihood that a tissue sample of unknown tissue origin is of spleen origin with respect to a given individual. In addition, with respect to a given individual, one could determine whether a given tissue sample of unknown origin is of spleen origin as opposed to having the origin of another tissue, e.g. the pancreas, prostate, or stomach. Such determinations may be used for various purposes including but not limited to the detection of tumor metastasis.

TABLE 1
Distribution of RNA coding for human SNORF138 receptor
using qRT-PCR
RNA encoding SNORF138 is expressed as copies/ng cDNA

| | | Potential therapeutic |
|---|---|---|
| Region | h-SNORF138 | applications |
| adipose (omental) | 121 | Metabolic disorders, |
| | | obesity, lipid metabolism |
| adipose (peri-renal) | 222 | Metabolic disorders, |
| | | obesity, lipid metabolism |
| adipose tissue | 129 | Metabolic disorders, |
| | | obesity, lipid metabolism |
| adrenal gland | 58 | Regulation of metabolic |
| (whole) | | steroids, regulation of |
| | | epinephrine release |
| amygdala | 35 | Depression, phobias, |
| | | anxiety, mood disorders |
| cerebellum | 31 | Motor coordination |
| cerebral cortex | 22 | Cognition, sensory and |
| | | motor integration |
| colon | 131 | Gastrointestinal |
| | | disorders |
| dorsal root ganglia | 31 | Sensory transmission, |
| | | pain |
| heart | 52 | Cardiovascular disorders |
| hippocampus | 31 | Cognition/memory |
| hypothalamus | 24 | Appetite/obesity, |
| | | neuroendocrine regulation |
| kidney cortex | 35 | Electrolyte balance, |
| | | hypertension |
| kidney medulla | 159 | Electrolyte balance, |
| | | hypertension |
| liver | 31 | Diabetes |
| lung | 155 | Respiratory disorders, |
| | | asthma |
| medulla | 14 | Sensory transmission |
| midbrain | 41 | Developmental disorders |
| pancreas | 7 | Diabetes, endocrine |
| | | disorders |
| pituitary (whole) | 13 | Endocrine/neuroendocrine |
| | | regulation |
| placenta | 393 | Reproductive function |
| pontine reticular | 7 | Regulation of |
| formation | | somatosensory, motor, |
| | | visual, auditory, |
| | | autonomic and affective |
| | | processes |
| prostate | 18 | Urinary dysfunction and |
| | | male sexual disfunction |
| skeletal muscle | 18 | Musculoskeletal disorders |
| small intestine | 73 | Gastrointestinal |
| | | disorders |
| spinal cord lumbar | 22 | Analgesia, sensory |
| | | modulation and |
| | | transmission |
| spleen | 352 | Immune disorders |
| stomach | 56 | Gastrointestinal |
| | | disorders |
| testes | 63 | Reproductive function, |
| | | regulation of steroid |
| | | hormones |
| thalamus | 16 | Sensory integration |
| | | disorders |
| uterus | 202 | Reproductive function |

Regulation of SNORF138 RNA in Diabetes.

The expression of SNORF138 was assayed in perirenal adipose tissue (five donors) and skeletal muscle (7 donors) obtained from diabetic doors. All donors were insulin dependant for a minimum of five years. The levels of SNORF138 in diabetic donors was compared to the levels detected in a pool of normal controls.

In the five peri-renal adipose samples assayed, SNORF138 is up-regulated (approximately 2-3 fold) in four of five donors. The fifth donor shows a modest decrease which is not distinguishable from the normal controls. Skeletal muscle from diabetic donors shows an inconsistent pattern of up-regulation. Four of the seven donors are indistinguishable from the normal pool, and the remaining three show a greater than four fold up-regulation over the normal pool.

These data, when considered with the expression profiling data, suggest that SNORF138 may play a role in diabetes. Its up-regulation in diabetic donors, combined with its expression profile indicate that the expression levels of this genes are altered in the disease state and compounds acting on this receptor may provide a methods for changing the clinical course of diabetes.

### REFERENCES

Bradford, M. M., “A rapid and sensitive method for the quantitation of microgram quantities of protein utilizing the principle of protein-dye binding”, Anal. Biochem. 72: 248-254 (1976).

Burns, C. C., et al., “Identification and deletion of sequences required for feline leukemia virus RNA packaging and construction of a high-titer feline leukemia virus packaging cell line”, Virology 222(1): 14-20 (1996).

Bush, et al., “Nerve growth factor potentiates bradykinin-induced calcium influx and release in PC12 cells”, J. Neurochem. 57: 562-574 (1991).

Chu, Y. Y., et al., “Characterization of the rat A2a adenosine receptor gene”, DNA Cell Biol. 15(4): 329-337 (1996).

Dascal, N., et al., “A trial G protein-activated K⁺ channel: expression cloning and molecular properties”, Proc. Natl. Acad. Sci. USA 90:10235-10239 (1993).

Fong, T. M., et al., “Mutational analysis of neurokinin receptor function” Can. J. Physio. Pharmacol. 73(7): 860-865 (1995).

Geib et al., “A novel Xenopus oocyte expression system based on cytoplasmic coinjection of T7-driven plasmids and purified T7-RNA polymerase.” Receptors & Channels. 7(5): 331-334, 2001.

Graziano, M. P. et al., “The amino terminal domain of the glucagon-like peptide-1 receptor is a critical determinant of subtype specificity” Receptors Channels 4(1): 9-17 (1996).

Guan, X. M., et al., “Determination of Structural Domains for G Protein Coupling and Ligand Binding in β3—Adrenergic Receptor” Mol. Pharmacol. 48(3): 492-498 (1995))

Jordan, M., Schallhorn, A., and Wurm, F. M. (1996) “Transfecting mammalian cells: optimization of critical parameters affecting calcium-phosphate precipitate formation.” Nucleic Acids Res. 24(4): 596-601.

Kubo, Y., et al., “Primary structure and functional expression of a rat G-protein-coupled muscarinic potassium channel” Nature 364:802-806 (1993).

Lazareno, S. and Birdsall, N. J. M., “Pharmacological characterization of acetylcholine stimulated [³⁵S]-GTPγS binding mediated by human muscarinic m1-m4 receptors: antagonist studies”, Br. J. Pharmacol. 109: 1120-1127 (1993).

Milligan, G. and Rees, S., “Chimeric G alpha proteins: Their potential use in Drug Discovery” Trends Pharmacol. Sci. 20: 118-124 (1999).

Quick, M. W. and Lester, H. A., “Methods for expression of excitability proteins in Xenopus oocytes”, Meth. Neurosci. 19: 261-279 (1994).

Salon, J. A. and Owicki, J. A., “Real-time measurements of receptor activity: Application of microphysiometric techniques to receptor biology” Meth. Neurosci. 25: 201-224 (1996).

Smith, K. E., et al., “Expression cloning of a rat hypothalamic galanin receptor coupled to phosphoinositide turnover”, J. Biol. Chem. 272: 24612-24616 (1997).

Spurney, R. F., et al., “The C-terminus of the thromboxane receptor contributes to coupling and desensitization in a mouse mesangial cell line”, J. Pharmacol. Exp. Ther. 283(1): 207-215 (1997).

Tian, W., et al., “Determinants of alpha-Adrenergic Receptor Activation of G protein: Evidence for a Pre-coupled Receptor/G protein State” Molecular Pharm. 45: 524-553 (1994)

Underwood, D. J. et al., “Structural model of antagonist and agonist binding to the angiotensin II, AT1 subtype, G protein coupled receptor”, Chem. Biol. 1(4): 211-221 (1994).

1-4. (canceled).

5. A process for determining whether a chemical compound is a human SNORF138 receptor antagonist which comprises contacting cells transfected with and expressing DNA encoding the human SNORF138 receptor, with the compound in the presence of a known human SNORF138 receptor agonist, under conditions permitting the activation of the human SNORF138 receptor, and detecting any decrease in human SNORF138 receptor activity, so as to thereby determine whether the compound is a human SNORF138 receptor antagonist.

6. (canceled).

7. A process for determining whether a chemical compound specifically binds to and inhibits activation of a human SNORF138 receptor, which comprises separately contacting cells producing a second messenger response and expressing on their cell surface the human SNORF138 receptor, wherein such cells do not normally express the human SNORF138 receptor, with both the chemical compound and a second chemical compound known to activate the human SNORF138 receptor, and with only the second chemical compound, under conditions suitable for activation of the human SNORF138 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the human SNORF138 receptor.

8. (canceled).

9. A method of screening a plurality of chemical compounds not known to inhibit the activation of a human SNORF138 receptor to identify a compound which inhibits the activation of the human SNORF138 receptor, which comprises:
1) contacting cells transfected with and expressing the human SNORF138 receptor with the plurality of compounds in the presence of a known human SNORF138 receptor agonist, under conditions permitting activation of the human SNORF138 receptor;
2) determining whether the extent or amount of activation of the human SNORF138 receptor is reduced in the presence of one or more of the compounds, relative to the extent or amount of activation of the human SNORF138 receptor in the absence of such one or more compounds; and if so
3) separately determining whether each such compound inhibits activation of the human SNORF138 receptor for each compound included in the plurality of compounds, so as to thereby identify any compound included in such plurality of compounds which inhibits the activation of the human SNORF138 receptor.

10. (canceled).

11. A process for preparing a pharmaceutical composition which comprises admixing a carrier, and a therapeutically effective amount of a chemical compound identified by the process of claim 5.

12. A process for preparing a pharmaceutical composition which comprises admixing a carrier, and a therapeutically effective amount of a chemical compound identified by the process of claim 7.

13. A process for preparing a pharmaceutical composition which comprises admixing a carrier, and a therapeutically effective amount of a chemical compound identified by the process of claim 9.

14-17. (canceled).
